(19) United States
(12) Patent Application Publication
(10) Pub. No.: US 2024/0240205 A1
(43) Pub. Date: Jul. 18, 2024

(54) METHODS AND COMPOSITION FOR INDUCING AN IMMUNE RESPONSE BY A RECOMBINNAT VACCINIA VIRUS

(71) Applicant: UNIVERSITY OF ROCHESTER, Rochester, NY (US)

(72) Inventors: Brian M. WARD, Pittsford, NY (US); Stephanie MONTICELLl, Frederick, MD (US); Peter BRYK, Rochester, NY (US)

(21) Appl. No.: 18/560,338

(22) PCT Filed: May 20, 2022
(86) PCT No.: PCT/US2022/030297

§ 371 Date: Nov. 10, 2023

Related U.S. Application Data
(60) Provisional application No. 63/202,295, filed on Jun. 4, 2021.

(51) Int. Cl. C12N 15/86; A61K 39/00; A61K 39/215; A61P 31/14

(52) U.S. Cl. CPC C12N 15/86; A61K 39/215; A61P 31/14; A61K 2039/5254; A61K 2039/5256

(57) ABSTRACT

A method for inducing an immune response to an antigen in a subject is disclosed. The method comprises the step of administering to the subject an effective amount of a recombinant vaccinia virus in which the coding sequence for the extracellular virion protein F13 has been replaced with the coding sequence for MC021, a molluscum contagiosum virus homolog of F13, wherein the recombinant vaccinia virus comprises a nucleic acid encoding an immunogenic epitope of the antigen.

This application is a U.S. National Stage Entry of PCT International Application No. PCT/US2022/030297, filed May 20, 2022, which claims priority from U.S. Application Ser. No. 63/202,295, filed Jun. 6, 2021. The entirety of the aforementioned application is incorporated herein by reference.

This invention was made with government support under Grant Nos. AI067391 and AI117105, awarded by the National Institute of Health. The government has certain rights in the invention.

### FIELD

This application relates in general to the field of medical treatment and, in particular, to the use of a recombinant vaccinia virus for inducing an immune response.

### BACKGROUND

Viral glycoproteins are a major component of the outermost envelope of viruses, actively participating in critical aspects of the viral lifecycle. The interaction between viruses and their hosts is often determined by the interactions between viral glycoproteins and host cell receptors, and thus deletion/mutation of glycoproteins found in the envelope of viruses often impact host cell entry, host range, and pathogen recognition. As such, viral glycoproteins are a major target of neutralizing antibodies. This combination of factors, in turn, affects viral pathogenesis.

Vaccinia virus (VACV), the prototypical member of the orthopoxvirus genus, produces two morphologically and antigenically distinct infectious forms of virions during its replication cycle: intracellular mature virions (IMV) and extracellular virions (EV). Following IMV production, a subset are trafficked to the trans-Golgi network, where two additional membranes are added to produce intracellular enveloped virions (IEV). IEV are a transient form that are transported to the cell surface, where fusion with the plasma membrane releases the EV form of the virus, which is critical for cell-to-cell spread and long-range dissemination. Four proteins found in EV and not IMV (A33, A34, B5 and F13), are highly conserved between members of the orthopoxvirus genus. Deletion of any one of these four proteins results in a small plaque phenotype, representative of their critical role for the efficient production of infectious EV. Of these four, deletion of F13 has the most profound effect on EV production due to defects in both EV production and infectivity.

VACV was a vital component of the largest and most successful vaccination program in history, producing protective immunity against smallpox, and is also widely used as the basis for many viral vaccine vectors, oncolytic vectors and gene therapy vectors. However, there are numerous complications arising from the use of VACV in patients, the majority of which stems from uncontrolled replication and spread of the virus from the original site of administration. Therefore, there is a need for safer, recombinant, or modified VACVs that maintain their immunogenicity as vaccine vectors, but have reduced pathogenicity, infectivity and side effects.

### SUMMARY

One aspect of the present application relates to a method for inducing an immune response to an antigen in a subject. The method comprises the step of administering to the subject an effective amount of a recombinant vaccinia virus in which the extracellular virion protein F13, (also known as: p37, 37-kDa protein, VACWR052, NCBI Gene ID 3707509) has been replaced with MC021, a molluscum contagiosum virus homolog of F13, wherein the recombinant vaccinia virus comprises a nucleic acid encoding an immunogenic epitope of the antigen.

In some embodiments, the antigen is a viral antigen. In some embodiments, the viral antigen is an antigen from SARS-Cov-2. In some embodiments, the antigen is a bacterial antigen. In some embodiments, the antigen is a tumor antigen.

In some embodiments, the recombinant vaccinia virus is administered percutaneously, subcutaneously or intramuscularly.

Another aspect of the present application relates to a method for inducing a protective immune response to a target in a subject. The method comprises the step of administering to the subject an effective amount of a recombinant vaccinia virus in which the extracellular virion protein F13 has been replaced with MC021, a molluscum contagiosum virus homolog of F13, wherein the recombinant vaccinia virus comprises a nucleic acid encoding an immunogen from the target.

In some embodiments, the target is a microorganism, such as viruses, bacteria, and parasites. In some embodiments, the target is SARS-Cov-2. In some embodiments, the target is a cell, such as tumor cell.

Another aspect of the present application relates to pharmaceutical composition comprising: (a) a recombinant vaccinia virus in which the extracellular virion protein F13 has been replaced with MC021, a molluscum contagiosum virus homolog of F13; and (b) a pharmaceutically acceptable carrier, wherein the pharmaceutical composition is formulated for or percutaneous inoculation, subcutaneous injection or intramuscular injection.

In some embodiments, the recombinant vaccinia virus comprises a nucleic acid encoding an immunogenic epitope of an antigen and is capable of expressing the epitope of the antigen upon infection of a target cell. In some embodiments, the antigen is an antigen from SARS-CoV-2

In some embodiments, the recombinant vaccinia virus is an enveloped virus and wherein the virus envelop comprises, in addition to MC021, an additional foreign glycoprotein or a portion thereof, wherein the additional foreign glycoprotein is a viral protein from a different virus. In some embodiments, the additional foreign glycoprotein is a viral protein from SARS-CoV-2.

### BRIEF DESCRIPTION OF THE DRAWINGS

While the present disclosure will now be described in detail, and it is done so in connection with the illustrative embodiments, it is not limited by the particular embodiments illustrated in the figures and the appended claims.

FIG. 1 is a schematic drawing of an embodiment of the recombinant vaccinia virus genome of the present application.

FIG. 2 shows glycoprotein content of EV membranes. (Panel A) RK13 cells were infected with the indicated viruses at a MOI of 5. At 24 hpi, extracellular virions were purified by CsCl density gradient centrifugation. Fractions from the gradient were collected dropwise from the bottom of the gradient and analyzed by OD260 and the concentration of CsCl determined by refractometry. (Panel B) Fractions containing EV were collected (left) and infected cells were harvested (right) and analyzed by Western blot with rabbit anti-HA antiserum followed by HRP-conjugated donkey anti-rabbit antibody, rat anti-B5 MAb followed by Alexa Fluor 647-conjugated donkey anti-rat antibody, rabbit anti-A33 antiserum followed by HRP-conjugated donkey-anti rabbit antibody, rabbit anti-L1 antiserum followed by Alexa Fluor 647-conjugated donkey anti-rabbit antibody, and mouse anti-actin MAb followed by Alexa Fluor 647-conjugated donkey anti-mouse antibody. The masses in kilodaltons and positions of marker proteins are shown on the right of the EV lysate blot and to the left of the cell lysate blot.

FIG. 3 shows a gating strategy for flow virometry. RK13 cells were infected with vF13L-HA at a MOI of 5 for 24 h. Extracellular virions were centrifuged through a sucrose cushion, fixed, and analyzed by flow cytometry by mCherry fluorescence intensity. The mCherry+population, denoting virions, was subsequently analyzed for glycoprotein incorporation. Events at the top end of the FSC (forward scatter) scale were excluded.

FIG. 4 shows EV glycoprotein content analyzed by flow virometry. (Panel A) RK13 cells were infected with the indicated viruses at a MOI of 5 and incubated at 37° C. overnight. The next day, EV were centrifuged through a sucrose cushion, fixed, and stained with rabbit anti-HA antiserum followed by Cy2-conjugated donkey anti-rabbit antibody, rat anti-B5 MAb followed by Dylight 405-conjugated donkey anti-rat antibody, and rabbit anti-A33 antiserum followed by Alexa Fluor 647-conjugated donkey anti-rabbit antibody. Shown are representative box and whisker plots for the incorporation of F13-HA/MC021-HA (left), B5 (middle), and A33 (right) in the EV membrane. ****, p<0.001 by Tukey's multiple comparison test following one-way ANOVA. (Panel B) Representative dot blots for the incorporation of B5 and F13-HA/MC021-HA (rows; top), A33 and F13-HA/MC021-HA (rows; middle), and A33 and B5 (rows; bottom) on viral surfaces for EV produced from vF13L-HA (columns; left), vMC021L-HA (columns; middle), and vΔF13L (columns right). All R2 values were significant (**** p<0.001).

FIG. 5 shows fluorescence activated virion sorting scheme. RK13 cells were infected with vF13L-HA/B5R-GFP at a MOI of 5 for 24 h. Extracellular virions were centrifuged through a sucrose cushion and analyzed by fluorescence-activated virion sorting. Virions were first gated on mCherry fluorescence intensity as in FIG. 3 and mCherry+ events were subsequently sorted based on GFP fluorescence intensity (B5GFPHIGH, B5GFPMED, and B5GFPLOW). Y-axis is count normalized for the number of sorted events (n) for easier visualization, and n is denoted for each population.

FIG. 6 shows B5-GFP recombinant virus plaque phenotype and flow virometry. (Panel A) Monolayers of BSC-40 cells were infected with the indicated viruses and incubated at 37° C. overnight. After 2 h, the inoculum was removed, and cells were overlaid with semisolid medium. After 3 days, cell monolayers were imaged by a fluorescent microscope and then subsequently stained with crystal violet and imaged. (Panel B) RK13 cells were infected with the indicated viruses at a MOI of 5 and incubated at 37° C. overnight. The next day, extracellular virions were centrifuged through a sucrose cushion, fixed, and stained with rabbit anti-HA antiserum followed by Cy5-conjugated donkey anti-rabbit antibody, rat anti-B5 MAb followed by Dylight 405-conjugated donkey anti-rat antibody, and rabbit anti-A33 antiserum followed by Alexa Fluor 750-conjugated goat anti-rabbit antibody. Shown are representative box and whisker plots for the incorporation of F13-HA/MC021-HA (left), B5 (middle left), A33 (middle right), and B5-GFP (right) in the EV membrane. ****, p<0.001 by Tukey's multiple comparison test following one-way ANOVA.

FIG. 7 shows fluorescence activated virion sorting and infectivity assays. RK13 cells were infected with the indicated viruses at a MOI of 5 and incubated at 37° C. overnight. The next day, EV were centrifuged through a sucrose cushion, and analyzed by fluorescence-activated virion sorting. Virions were sorted into 3 pools based on GFP fluorescence intensity (B5GFPHIGH, B5GFPMED, and B5GFPLOW) and analyzed by qPCR for (Panel A) total genome copies and (Panel B) cell binding. (Panel C) Specific infectivity was calculated by comparing total genome copies (by qPCR) and plaque forming units (by plaque assay). **, p<0.01 and ****, p<0.001 by Tukey's multiple comparison test following one-way ANOVA.

FIG. 8 shows pathogenesis and Immunity. (Panel A-F) Wild-type C57Bl/6 mice were infected with 10,000 pfu of the indicated viruses in a single ear pinna and tissue pathology visualized (Panel A-C), and tissue swelling (Panel D), development of a lesion at the site of infection (Panel E) and tissue loss at the site of infection (Panel F) was measured. Graphs showed pooled data from two experiments (n=10). (Panel G) STAT1−/− mice (n≥3) were infected intradermally with 10,000 pfu of the indicated viruses in each ear pinna and survival was monitored for 28 days post-infection. Graph is representative of pooled data from two experiments (n=7-10). (Panel H) Wild-type C57Bl/6 mice were immunized with 10,000 pfu of the indicated viruses in a single ear pinna, serum harvested 38 days later and the ability to serum dilutions to inhibit infection with VACV-GFP assayed by flow cytometry. (Panel I) Wild-type C57Bl/6 mice were immunized with 10,000 pfu of the indicated viruses in a single ear pinna, and the splenic CD8+ T cell responses to immunodominant B8, A8, A3, K3, A47 and A42 determinants assayed by conventional intracellular cytokine secretion assay. (Panel J) Wild-type Balb/c mice were immunized with 10,000 pfu of the indicated viruses in a single ear pinna, then 35 days later challenged by footpad infection with 3000 pfu of the virulent mouse pathogen, ECTV. Survival of mice is shown from two combined experiments (n=10).

### DETAILED DESCRIPTION

Herein incorporated by reference is the sequence listing filed with the USPTO named as 1134-070 PCT.txt which was created on May 20, 2022, and the size of the TXT file is 7,112 in bytes.

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan. For example, if the value “10” is disclosed the “less than or equal to 10” as well as “greater than or equal to 10” is also disclosed.

### I. Definitions

The term “vaccinia virus” as used herein refers a large, complex, enveloped virus belonging to the poxvirus family. It has a linear, double-stranded DNA genome approximately 190 kbp in length, and which encodes approximately 200 proteins. Vaccinia virus strains include, but are not limited to, strains of, derived from, or modified forms of Western Reserve (WR), Copenhagen, Tashkent, Tian Tan, Lister, Wyeth, IHD-J, and IHD-W, Brighton, Ankara, MVA, Dairen I, LIPV, LC16M8, LC16MO, LIVP, WR 65-16, Connaught, New York City Board of Health vaccinia virus strains.

A “recombinant vaccinia virus” refers to a vaccinia virus containing one or more heterogeneous nucleotide, replicates heterogeneous nucleotide or expresses said nucleotide, a peptide, a heterogeneous peptide, or a protein encoded by a heterogeneous nucleotide in the virus infected cells. The recombinant virus can express a gene or a gene fragment in a sense or antisense form, which are not found in the natural state of the virus. In addition, the recombinant virus can express a gene which is found in a virus of a natural state. However, the gene is a modified gene, re-introduced or rescued into a gene of the virus by an artificial means.

As used herein, the term “immune response” refers to a response of a cell of the immune system, such as a B cell, T cell, dendritic cell, macrophage or polymorphonucleocyte (PMN), to a stimulus, such as an antigen or vaccine. An immune response can include any cell of the body involved in a host defense response, including for example, an epithelial cell that secretes an interferon or a cytokine. An immune response includes, but is not limited to, an innate and/or adaptive immune response. Methods of measuring immune responses are well known in the art and include, for example, measuring proliferation and/or activity of lymphocytes (such as B or T cells), measuring secretion of cytokines or chemokines, inflammation, antibody production and the like.

The terms “protective immune response” and “protective immunity” refer to an immune response or state of immunity in which a subject's immune system can facilitate protection in a subject from an infection (e.g., prevents infection or prevents the development of disease associated with infection) or disease state characterized by the presence of one or more antigens ordinarily foreign to a host.

The terms “antigen” refers to a substance or molecule capable of eliciting an immune response and generating specific antibodies (humoral response) or cytotoxic T-lymphocytes (cell-mediated response) against it. As such, the antigen or immunogen is capable of being recognized by components of the immune system, such as antibodies or lymphocytes. An antigen can be as small as a single epitope, or larger, and can include multiple epitopes. As such, the size of an antigen can be as small as about 5-12 amino acids (e.g., a peptide) and as large as: a partial protein, a full length protein, including a multimer and fusion protein, chimeric protein, or agonist protein or peptide. In addition, antigens can include carbohydrates. The antigen or immunogen may be a microorganism (or pathogen)-derived antigen, a neoantigen, a tumor cell antigen or a self-antigen. A microorganism-derived (or pathogen-derived) antigen/immunogen may be from a bacterium, virus, protozoan or fungus.

The term “immunogen” refers to a molecule having the ability to be recognized by immunological receptors such as T cell receptor (TCR) or B cell receptor (BCR or antibody). The immunogen may be natural or non-natural, provided it presents at least one epitope, for example a T cell and/or a B cell epitope.

The term “immunogenic” refers to a reaction triggered by the presence of an epitope of an antigen or immunogen. The term “epitope” refers to an antigenic determinant that is sufficient to elicit an immune response. These are particular chemical groups or peptide sequences on a molecule that are antigenic, such that they elicit a specific immune response, for example, an epitope is the region of an antigen to which B and/or T cells respond. Epitopes can be formed both from contiguous amino acids or noncontiguous amino acids juxtaposed by tertiary folding of a protein. Those of skill in the art will recognize that T cell epitopes are different in size and composition from B cell epitopes, and that epitopes presented through the Class I MHC pathway differ from epitopes presented through the Class II MHC pathway.

The terms “tumor antigen”, “tumor-specific antigen” and “tumor-associated antigen” thereof are used with reference to an antigen associated with a preneoplastic state, hyperplastic state, or neoplastic state. Such antigens may also be associated with, or a causative agent of cancer.

The term “neoantigen” is used herein with reference to an antigen that has at least one alteration making it distinct from the corresponding wild-type, parental antigen, e.g., via mutation in a tumor cell or post-translational modification specific to a tumor cell. The alteration may be the result of a mutation in a subject's DNA, such as a frameshift, nonframeshift indel (e.g., small insertions or deletions (e.g., of nucleotides) or substitution polymorphisms (indels), missense or nonsense substitutions, splice site alterations, genomic rearrangements or gene fusions, splice variants, aberrant phosphorylation or glycosylation and the like. In certain embodiments, the neoantigen is a tumor antigen.

Tumor antigens include, but are not limited to, antigens from any tumor or cancer, including, but not limited to, melanomas, squamous cell carcinoma, breast cancers, head and neck carcinomas, thyroid carcinomas, soft tissue sarcomas, bone sarcomas, testicular cancers, prostatic cancers, ovarian cancers, bladder cancers, skin cancers, brain cancers, angiosarcomas, hemangiosarcomas, mast cell tumors, leukemias, lymphomas, primary hepatic cancers, lung cancers, pancreatic cancers, gastrointestinal cancers (including colorectal cancers), renal cell carcinomas, hematopoietic neoplasias and metastatic cancers thereof.

The term “vaccine” refers to a composition that induces an immune response in the recipient or host of the vaccine. The vaccine may induce a humoral (e.g., neutralizing antibody) response to one or more antigens, cell-mediated immune response (e.g., cytotoxic T lymphocyte (CTL)) response against one or more antigens, or both in a recipient so as to provide partial or complete protection against e.g., current or subsequent microbial infections or disease conditions characterized by the presence of e.g., one or more neoantigens or cancer antigens in the recipient. Examples of vaccines include, but are not limited to, live attenuated vaccines, inactivated vaccines, subunit vaccines (protein or peptide), sugar based vaccines (conjugated or natural), bacterial vector vaccines and viral vector vaccines.

The term “vaccination” refers to the administration of antigenic material to stimulate an individual's immune system to develop adaptive immunity to a pathogen or a host cell containing a non-natural antigen in a host. Vaccination can prevent or ameliorate of one or more symptoms associated with microbial infection or antigen- or epitope-specific cell associated with a disease, such as cancer; and/or lessening of the severity or frequency of one or more symptoms associated with the foregoing disease conditions.

The term “immunize” is used with reference to rendering a subject protected from an infectious disease or disease state, such as by vaccination.

The terms “protection”, “immune protection” and “protective response” are used interchangeably to convey partial or complete resistance to subsequent infections, active infections or certain disease conditions in a host. Neutralizing antibodies generated in a vaccinated host can provide this protection. In other situations, CTL responses can provide this protection. In some situations, both neutralizing antibodies and cell-mediated immune (e.g., CTL) responses provide this protection.

The term “adjuvant” refers to an agent that when administered concurrently with the vaccine composition of the present application, accelerates, prolongs, enhances and/or boosts the immune response thereto. Adjuvants can enhance an immune response by several mechanisms including, e.g., lymphocyte recruitment, stimulation of B and/or T cells, stimulation of dendritic cells and/or stimulation of macrophages.

The terms “control sequences” or “regulatory sequences” refer to DNA sequences refer to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell. Control/regulatory sequences include those which direct constitutive expression of a nucleotide sequence in many types of host cells and those which direct expression of the nucleotide sequence only in certain host cells (e.g., tissue-specific regulatory sequences).

A nucleic acid sequence is “operably linked” to another nucleic acid sequence when the former is placed into a functional relationship with the latter. Generally, “operably linked” means that the DNA or RNA sequences being linked are contiguous and, in the case of a secretory leader, contiguous and in reading phase. Further, a promoter or enhancer is said to be operably linked to a coding sequence if it affects the transcription of the sequence and a ribosome binding site is operably linked to a coding sequence if it is positioned so as to facilitate translation. However, enhancers do not have to be contiguous. An expression vector may further include an intron or chimeric intron.

As used herein, the term “promoter” is to be taken in its broadest context and includes transcriptional regulatory elements (TREs) from genomic genes or chimeric TREs therefrom, including the TATA box or initiator element for accurate transcription initiation, with or without additional TREs (i.e., upstream activating sequences, transcription factor binding sites, enhancers and silencers) which regulate activation or repression of genes operably linked thereto in response to developmental and/or external stimuli and trans-acting regulatory proteins or nucleic acids. The promoter may be constitutively active or it may be active in one or more tissues or cell types in a developmentally regulated manner. A promoter may contain a genomic fragment or it may contain a chimera of one or more TREs combined together.

The phrases “to a patient in need thereof”, “to a patient in need of treatment” or “a subject in need of treatment” includes subjects, such as mammalian subjects, that would benefit from administration of the nucleic acid and protein immunogens of the present disclosure for vaccination against a microorganism.

The terms “therapeutically effective amount”, “pharmacologically effective amount”, and “physiologically effective amount” are used interchangeably to mean the amount of a vaccine composition needed to provide a threshold level of immune protection. The precise amount will depend upon numerous factors, e.g., the particular immunogens utilized, the components and physical characteristics of the composition, intended patient population, patient considerations, and the like, and can readily be determined by one skilled in the art, based upon the information provided herein or otherwise available in the relevant literature.

The terms, “improve”, “increase” or “reduce”, as used in this context, indicate values or parameters relative to a baseline measurement, such as a measurement in the same individual prior to initiation of the treatment described herein, or a measurement in a control individual (or multiple control individuals) in the absence of the treatment described herein.

### II. Methods for Inducing an Immune Response

One aspect of the application relates to a method for inducing an immune response to a target antigen in a subject. The method comprises the step of administering to the subject an effective amount of a recombinant vaccinia virus in which the extracellular virion protein F13 has been replaced with MC021, a molluscum contagiosum virus homolog of F13, wherein the recombinant vaccinia virus comprises a nucleic acid encoding an immunogenic epitope of the target antigen. The complete amino acid sequence of F13 and MC021 are listed below:

Vaccinia virus F13:
(SEQ ID NO: 1)
MWPFASVPAGAKCRLVETLPENMDFRSDHLTTFECFNEIITLAKKYIYI
ASFCCNPLSTTRGALIFDKLKEASEKGIKIIVLLDERGKRNLGELQSHC
PDINFITVNIDKKNNVGLLLGCFWVSDDERCYVGNASFTGGSIHTIKTL
GVYSDYPPLATDLRRRFDTFKAFNSAKNSWLNLCSAACCLPVSTAYHIK
NPIGGVFFTDSPEHLLGYSRDLDTDVVIDKLRSAKTSIDIEHLAIVPTT
RVDGNSYYWPDIYNSIIEAAINRGVKIRLLVGNWDKNDVYSMATARSLD
ALCVQNDLSVKVFTIQNNTKLLIVDDEYVHITSANFDGTHYQNHGFVSF
NSIDKQLVSEAKKIFERDWVSSHSKSLKI
MC021:
(SEQ ID NO: 2)
MGNLTSARPAGCKIVETLPATLPLALPTGSMLTYDCFDTLISQTQRELC
IASYCCNLRSTPEGGHVLLRLLELARADVRVTIIVDEQSRDADATQLAG
VPNLRYLKLDVGELPGGKPGSLLSSFWVSDKRRFYLGSASLTGGSISTI
KSLGVYSECEPLARDLRRRFRDYERLCARRCVRCLSLSTRFHLRRHCEN
AFFSDAPESLIGSTRTFDADAVLAHVQAARSTIDMELLSLVPLVRDEDS
VQYWPRMHDALVRAALERNVRVRLLVGLWHRSDVFSLAAVKGLHELGVG
HADISVRVFAIPGAKGDAVNNTKLLVVDDEYVHVTSADMDGTHYARHAF
VSFNCAERAFARALGALFERDWQSSFSSPLPRAPPPEPATLLPVN

In some embodiments, the target antigen is a viral antigen. In some embodiments, the target antigen is a bacterial antigen. In some embodiments, the target antigen is a tumor antigen.

Another aspect of the application is a method for inducing a protective immune response to a target microorganism or a target cell in a subject. The method comprises the step of administering to the subject an effective amount of a recombinant vaccinia virus in which the extracellular virion protein F13 has been replaced with MC021, wherein the recombinant vaccinia virus comprises a nucleic acid encoding an immunogen or antigen from the target.

In some embodiments, the target microorganism is a virus. In some embodiments, the target microorganism is a bacterium. In some embodiments, the target microorganism is a parasite. In some embodiments, the target microorganism is a fungus. In some embodiments, the target cell is a tumor cell.

### Targets of Immune Response

The target antigen, target microorganism or target cell can be any antigen, microorganism or cell, where an immune response against the antigen, microorganism or cell is desired or needed. In some embodiments, the target antigen is derived from the target microorganism or target cell. In some embodiments, the target antigen comprises a polypeptide which is a region within the surface protein of the target microorganism or target cell. In some embodiments, the polypeptide comprises an epitope known to one of ordinary skill in the art for inducing immune response or protective immune response in a subject.

In some embodiments, the methods of the present application is used for vaccination against a target virus. The target virus is an RNA virus. Exemplary RNA viruses for vaccination include retroviruses (e.g., HIV-1, HIV-2, HTLV-I, HTLV-II); bunyaviruses (e.g., Rift Valley fever virus, Crimean-Congo hemorrhagic fever virus); filoviruses (e.g., Ebola virus, Marburg virus); flaviviruses (e.g., Hepatitis C virus, West Nile virus, Dengue fever virus, Zika virus, yellow fever virus, tick-borne encephalitis virus, Saint Louis encephalitis virus, GB virus C); enteroviruses (Types A to L, including coxsackieviruses (Types A to C), echoviruses, rhinoviruses (Types A to C), poliovirus); orthomyxoviruses (e.g., influenza Types A, -B, -C, -D, including A subtypes H1N1, H5N1, H3N2); paramyxoviruses (e.g., rubulavirus (mumps), rubeola virus (measles), respiratory syncytial virus, Newcastle disease, parainfluenza); parvoviruses (e.g., parvovirus B19 virus); rhabdoviruses (e.g., Rabies virus); arenaviruses (e.g., lymphocytic choriomeningitis virus and several Lassa fever viruses, including Guanarito virus, Junin virus, Lassa virus, Lujo virus, Machupo virus, Sabia virus, Whitewater Arroyo virus); alphaviruses (e.g., Venezuelan equine encephalitis virus, eastern equine encephalitis virus; western equine encephalitis virus); Hepatitis A virus; Hepatitis D virus; Hepatitis E virus; as well as any type, subtype, clade or sub-clade thereof.

In some embodiments, the RNA virus is a coronavirus (CoV) in the Orthocoronavirinae family. The genetically diverse Orthocoronavirinae family is divided into four genera (alpha, beta, gamma, and delta coronaviruses). The four genera are further divided into subgroup 1a alphacoronaviruses, subgroup 1b alphacoronaviruses, subgroup 2a betacoronaviruses, subgroup 2b betacoronaviruses, subgroup 2c betacoronaviruses and subgroup 2d betacoronaviruses.

Human CoVs are limited to the alpha and beta subgroups. Exemplary human CoVs for vaccination include severe acute respiratory syndrome coronavirus-2 (SARS-CoV-2), severe acute respiratory syndrome coronavirus (SARS-CoV), Middle East respiratory syndrome coronavirus (MERS-CoV), HCoV-229E, HCoV-OC43, HCoV-NL63, and HCoV-HKU1.

In some embodiments, the RNA virus for vaccination is a respiratory virus, such as influenza Type A virus. Influenza A viruses are divided into subtypes on the basis of two proteins on the surface of the virus, hemagglutinin (HA) and neuraminidase (NA). There are 18 known HA subtypes and 11 known NA subtypes. Many different combinations of HA and NA proteins are possible. For example, an “H7N2 virus” designates an influenza A virus subtype that has an HA 7 protein and an NA 2 protein. Similarly, an “H5N1” virus has an HA 5 protein and an NA 1 protein. Type A influenza viruses that may be targeted for vaccination according to the methods and compositions of the present disclosure, which can be used to target a variety of sub-types, such as H1N1, H3N2, H5N1, H5N2, H5N3, H5N4, H5N5, H5N6, H5N7, H5N8, and H5N9, H7N1, H7N2, H7N3, H7N4, H7N5, H7N6, H7N7, H7N8, and H7N9, H9N1, H9N2, H9N3, H9N4, H9N5, H9N6, H9N7, H9N8, H9N9, H17N10, H18N11 and combinations thereof.

In some embodiments, the virus for vaccination is a DNA virus. Exemplary DNA viruses for vaccination include herpesviruses (e.g., HSV-1, HSV-2, EBV, VZV, HCMV-1, HHV-6, HHV-7, HHV-8), papillomaviruses (e.g., human papilloma virus (HPV) Types 1, 2, 4, 6, 11, 16, 18, 26, 30, 31, 33, 34, 35, 39, 40, 41, 42, 43, 44, 45, 51, 52, 54, 55, 56, 57, 58, 59, 61, 62, 64, 67, 68, 69, 70); poxviruses (e.g., smallpox virus), hepadnaviruses (Hepatitis B virus); anelloviruses (e.g., transfusion transmitted virus or torque teno virus (TTV)); as well as any type, subtype, clade or sub-clade thereof.

In some embodiments, the methods of the present application is used to introduce immune responses against a tumor antigen or a tumor cell. As used herein, the terms “tumor antigen”, “tumor-specific antigen” and “tumor-associated antigen” thereof are used with reference to an antigen associated with a preneoplastic state, hyperplastic state, or neoplastic state. Such antigens may also be associated with, or a causative agent of cancer. The term “neoantigen” refers to an antigen that has at least one alteration making it distinct from the corresponding wild-type, parental antigen, e.g., via mutation in a tumor cell or post-translational modification specific to a tumor cell. The alteration may be the result of a mutation in a subject's DNA, such as a frameshift, nonframeshift indel (e.g., small insertions or deletions (e.g., of nucleotides) or substitution polymorphisms (indels), missense or nonsense substitutions, splice site alterations, genomic rearrangements or gene fusions, splice variants, aberrant phosphorylation or glycosylation and the like. In certain embodiments, the neoantigen is a tumor antigen.

Tumor antigens include, but are not limited to, antigens from any tumor or cancer, including, but not limited to, melanomas, squamous cell carcinoma, breast cancers, head and neck carcinomas, thyroid carcinomas, soft tissue sarcomas, bone sarcomas, testicular cancers, prostatic cancers, ovarian cancers, bladder cancers, skin cancers, brain cancers, angiosarcomas, hemangiosarcomas, mast cell tumors, leukemias, lymphomas, primary hepatic cancers, lung cancers, pancreatic cancers, gastrointestinal cancers (including colorectal cancers), renal cell carcinomas, hematopoietic neoplasias and metastatic cancers.

### Recombinant Vaccinia Virus

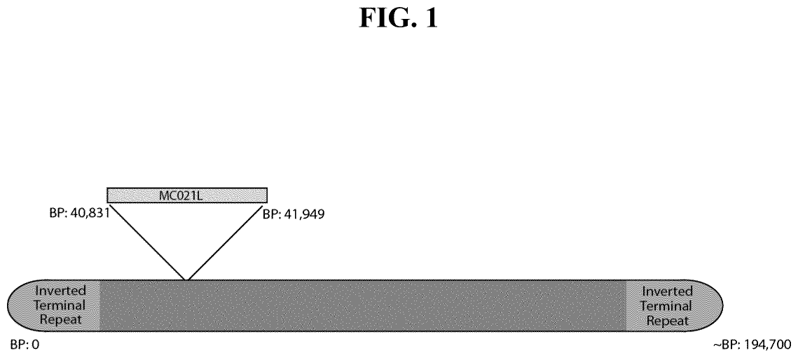

The recombinant vaccinia virus of the present application is a vaccinia virus in which the coding sequence for extracellular virion protein F13 has been replaced with the coding sequence for MC021, a molluscum contagiosum virus homolog of F13. Methods for preparing such a recombinant vaccinia virus are well known in the art. An exemplary procedure for generating such a recombinant vaccinia virus is described in Example 1. The recombinant vaccinia virus can be generated from any vaccinia virus strains. In some embodiments, the recombinant vaccinia virus is derived from the group consisting of vaccinia Copenhagen, ACAM 2000, ACAM 3000, International Health Department (IID) vaccinia strains, vaccinia Lister, defective vaccinia Lister, vaccinia Wyeth, vaccinia Ankara, modified vaccinia Ankara (MVA), MVA-575 (ECACC V00120707), MVA-BN (ECACC V00083008) and the New York Board of Health vaccinia strains. FIG. 1 is a diagrammatic representation of the recombinant vaccinia virus genome. Shown is the approximate location of VACWR052, also known as F13L, that was removed and replaced with the MC021L gene from molluscum contagiosum virus.

In some embodiments, the recombinant vaccinia virus comprises a nucleotide sequence encoding an antigen/immunogen from a target microorganism or a target cell, a fragment of the antigen/immunogen and/or an epitope of the antigen/immunogen. The coding sequence is operably linked to a regulatory element that controls the expression of the coded antigen/immunogen and/or fragments/epitopes thereof. In some embodiments, the regulatory element is a poxvirus promoter. Typically, the coding sequence and the regulatory element are inserted in an unobtrusive region, such as any intergenic region. Alternatively, coding sequences can replace genes that are not essential to virus replication and morphogenesis. Examples include, but are not limited to, the thymidine kinase, hemagglutinin, vaccinia virus growth factor, ribonucleotide reductase subunits genes (J2R, A56R, C11R, F4L & I4L, respectively). These chimeric genes could then be placed under a poxvirus promoter and inserted into the genome in an unobtrusive region.

In some embodiments, the recombinant vaccinia virus of the present application is configured to express a target glycoprotein antigen on the envelope of released vaccinia virus, so as to induce an immune response against the target glycoprotein antigen in a host after inoculation of the recombinant vaccinia virus. In some embodiments, foreign glycoproteins (e.g., glycoproteins from other viruses) are targeted to the envelope of the recombinant vaccinia virus by fusing the coding sequence of the extracellular domain of the foreign glycoproteins to the coding sequence of the transmembrane and cytoplasmic tail domain of either A33 and A34 (for type II glycoproteins) or B5 (for type I glycoproteins). These chimeric genes could then be placed under a strong poxvirus late promoter and inserted into the genome in an unobtrusive region. Examples of the target glycoprotein antigens include, but are not limited to, influenza (A and B) hemagglutinin/neuraminidase, Coronavirus Spike protein, HIV gp120, Flavivirus' (Zika virus, Yellow fever virus, West Nile virus, Dengue virus) E protein, Human Herpes virus (HHV1, HHV2, HHV3, HHV4, HHV5, HHV6, HHV7 & HHV8) and glycoproteins gB, gD, gO, gM, gN, and gHgL.

### Dosages and Route of Administration

The recombinant vaccinia virus of the present application can be administered by any route of administration, so long as the recombinant virus is capable of triggering a desired immune response after administration. In some embodiments, the recombinant vaccinia virus of the present application is administered by percutaneous inoculation (scarification of the skin). In some embodiments, the recombinant vaccinia virus of the present application is administered by subcutaneous administration. In some embodiments, the recombinant vaccinia virus of the present application is administered by intramusclular administration.

In some embodiments, the recombinant vaccinia virus of the present application is administered at a dose in the range of 1×10⁴-1×10⁸ PFU. In some embodiments, the recombinant vaccinia virus of the present application is administered at a dose in the range of 1×10⁴-1×10⁵ PFU, 1×10⁴-3×10⁵ PFU, 1×10⁴-1×10⁶ PFU, 1×10⁴-3×10⁶ PFU, 1×10⁴-1×10⁷ PFU, 1×10⁴-3×10⁷ PFU, 3×10⁴-1×10⁵ PFU, 3×10⁴-3×10⁵ PFU, 3×10⁴-3×10⁶ PFU, 3×10⁴-3×10⁶ PFU, 3×10⁴-1×10⁷ PFU, 3×10⁴-3×10⁷ PFU, 3×10⁴-1×10⁸ PFU, 1×10⁵-3×10⁵ PFU, 1×10⁵-1×10⁶ PFU, 1×10⁵-3×10⁶ PFU, 1×10⁵-1×10⁷ PFU, 1×10⁵-3×10⁷ PFU, 1×10⁵-1×10⁸ PFU, 3×10⁵-1×10⁶ PFU, 3×10⁵-3×10⁶ PFU, 3×10⁵-1×10⁷ PFU, 3×10⁵-3×10⁷ PFU, 3×10⁵-1×10⁸ PFU, 1×10⁶-3×10⁶ PFU, 1×10⁶-1×10⁷ PFU, 1×10⁶-3×10⁷ PFU, 1×10⁶-1×10⁸ PFU, 3×10⁶-1×10⁷ PFU, 3×10⁶-3×10⁷ PFU, 3×10⁶-1×10⁸ PFU, 1×10⁷-3×10⁷ PFU, 1×10⁷-1×10⁸ PFU or 3×10⁷-1×10⁸ PFU.

In some embodiments, the recombinant vaccinia virus of the present application is administered percutaneously, subcutaneously or intramuscularly at a dose in the range of 5×10⁵-2×10⁶ PFU. In some embodiments, the recombinant vaccinia virus of the present application is administered percutaneously, subcutaneously or intramuscularly at a dose of 1×10⁶ PFU.

In some embodiments, the dose described above is administered 2, 3, 4 or 5 times with an interval of 1, 2, 3, 4, 5, 6 or 7 days. In some embodiments, the dose described above is administered 2, 3, 4 or 5 times with an interval of 1, 2, 3, 4, 5, 6 or 7 weeks. In some embodiments, the dose described above is administered 2, 3, 4 or 5 times with an interval of 1, 2, 3, 4, 5, 6 or 7 months. In some embodiments, a single dose is split into multiple injections. For example, a single dose of 1×10⁶ PFU may be split into two doses of 5×10⁵ PFU each and administered with an interval of 1, 2, 3, 4, 5, 6 or 7 days.

In some embodiments, the dosing regimen is modified based on the size, weight, age and sex of the patient, the nature and stage of the disease, the aggressiveness of the disease, and the route of administration of the composition.

### III. Combination Therapy

In some embodiments, the recombinant vaccinia virus of the present application is administered in combination with one or more additional agents. In some embodiments, the one or more additional agent comprises an antiviral agent, such as an antiviral agent against coronaviruses. Exemplary antiviral agents include, but are not limited to, viral polymerase inhibitors, such as Remdesivir, GS-441524, Faviravir, EIDD-2801, EIDD-2901, EIDD-1931, Ribavirin, 6-azauridine; convalescent plasma; neutralizing mAbs or mAbs inhibiting coronavirus attachment or entry, such as REGN10933, REGN10987, LY3819253, AZD7442, BRII-196, CT-P59, JS016, SCTA01, STI-1499, TY027, 47D11; anti-IL6 monoclonal antibodies, such as Tocilizumab; protease inhibitors targeting Mpro, such as Lopinavir, Ritonavir, Dipyridamole, and Danoprevir; Ivermectin; Saracatinib; protease inhibitors targeting TMPRSS2, such as Camostat, Nafomastat, and Nafomastat mesylate; S-protein targeted drugs, such as Arbidol (umifenovir) and Hydroxychloroquine; cytokines, such as interferons, Dexamethasone, Anakinra, Hydrocortisone, Azithromycin, Ulinastatin and Ciclesonide; Janus kinase inhibitors, such as Ruxolitinib and Baricitinib; AXL kinases inhibitors, such as Bemcentinib; Dihydroorotate dehydrogenase (DHODH) inhibitors, such as PTC299; recombinant ACE2; HR2P-EK1C4, IPBO3 and other lipopeptide fusion inhibitors described herein above; Fluvoxamine; Apilomod; Ciclesonide; Tetrahydroquinoline oxocarbazate; GC373; Vitamin D; Zn2+; and combinations thereof.

Additional antiviral agents for use in combination with the composition of the present application include, but are not limited to, abacavir, acyclovir, adefovir, amantadine, amdoxovir, amprenavir, antiprotease, apricitabine, arbidol, artemisinin, atazanafir, atripla, azidothymidine (AZT), azithromycin, bevirimat, boceprevir, butylated hydroxytoluene (BHT), chloroquine, cidofovir, combivir, darunavir, delavirdine, didanosine, dipivoxil, docosanol, edoxudine, efavirenz, elvitegravir, elvucitabine, emtricitabine, enfuviritide, entecavir, etravirine, famciclovir, foscarnet, fosamprenavir, gancyclovir, globoidnan A, GSK-572, HIV fusion inhibitors, hydroxychloroquine, hypericin, ibalizumab, idoxuridine, immunovir, indinavir, interferons (Types I, II and III), lamivudine, lersivirine, lopinivir, loviride, maraviroc, maribavir, MK-2048, molixan (NOV-205), moroxydine, nelfinavir, nevirapine, nexavir, non-nucleotide HIV RT inhibitors, oseltamivir, pegylated interferons (e.g., peginterferon alfa-2a), penciclovir, pencyclovir, peramivir, pleconaryl, podophyllotoxin, racivir, raltegravir, remdesivir, resquimod, ribavirin, rifampin, rilpivirine, rimantidine, ritonavir, saquinivir, stampidine, stavudine, taribavirin, tenofovir, tipranavir, trifluridine, trizivir, tromantidine, truvada, valaciclovir (Valtrex), valacyclovir, valganciclovir, vicriviroc, vidarabine, vivecon, zalcitabine, zanamivir (Relenza), zidovudine, zinc sulfate, and combinations thereof.

In some embodiments, the recombinant vaccinia virus of the present application is administered in combination with one or more additional anti-cancer agents. The anti-cancer agent may be an alkylating agent; an anthracycline antibiotic; an anti-metabolite; a detoxifying agent; an interferon; a polyclonal or monoclonal antibody; a check-point regulator (e.g., PD-1 or PDL-1 or CTL-4 inhibitor); an EGFR inhibitor; a HER2 inhibitor; a histone deacetylase inhibitor; a hormone or anti-hormonal agent; a mitotic inhibitor; a phosphatidylinositol-3-kinase (PI3K) inhibitor; an Akt inhibitor; a mammalian target of rapamycin (mTOR) inhibitor; a proteasomal inhibitor; a poly(ADP-ribose) polymerase (PARP) inhibitor; a Ras/MAPK pathway inhibitor; a centrosome declustering agent; a multi-kinase inhibitor; a serine/threonine kinase inhibitor; a tyrosine kinase inhibitor; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase or a protein methyltransferase), a cytidine analogue or combination thereof.

### IV. Pharmaceutical Compositions

Another aspect of the present application relates to a pharmaceutical composition. The pharmaceutical composition comprises the recombinant vaccinia virus of the present application (i.e., a recombinant vaccinia virus in which the extracellular virion protein F13 has been replaced with MC021, a molluscum contagiosum virus homolog of F13) and a pharmaceutically acceptable carrier. In some embodiments, the pharmaceutical composition is formulated for subcutaneous injection or dermal inoculation.

As used herein, the term “pharmaceutically acceptable” refers to a molecular entity or composition that does not produce an adverse, allergic or other untoward reaction when administered to an animal or a human, as appropriate. The term “pharmaceutically acceptable carrier”, as used herein, includes any and all solvents, solubilizers, fillers, stabilizers, surfactants, binders, absorbents, bases, buffering agents, excipients, lubricants, controlled release vehicles, diluents, emulsifying agents, humectants, lubricants, gels, dispersion media, coatings, antibacterial or antifungal agents, isotonic and absorption delaying agents, and the like, which are compatible with pharmaceutical administration. In certain embodiments, the pharmaceutically acceptable carrier comprises serum albumin. The use of such carriers and agents for pharmaceutically active substances is well known in the art.

Exemplary carriers or excipients include but are not limited to, calcium carbonate, calcium phosphate, various sugars, starches, cellulose derivatives, gelatin, polymers such as polyethylene glycols, water, saline, isotonic aqueous solutions, phosphate buffered saline, dextrose, 0.3% aqueous glycine, glycerol, ethanol and the like, as well as combinations thereof. In many cases, it will be preferable to include isotonic agents, for example, sugars, polyalcohols such as mannitol, sorbitol, or sodium chloride in the composition, or glycoproteins for enhanced stability, such as albumin, lipoprotein and globulin. Pharmaceutically acceptable carriers may further comprise minor amounts of auxiliary substances such as wetting or emulsifying agents, preservatives or buffers, which enhance the shelf life or effectiveness of the therapeutic agents. In certain embodiments, the pharmaceutically acceptable carrier comprises serum albumin.

Formulation characteristics that can be modified include, for example, pH and osmolality. For example, it may be desired to achieve a formulation that has a pH and osmolality similar to that of human blood or tissues to facilitate the formulation's effectiveness when administered parenterally.

Buffers are useful in the present disclosure for, among other purposes, manipulation of the total pH of the pharmaceutical formulation (especially desired for parenteral administration). A variety of buffers known in the art can be used in the present formulations, such as various salts of organic or inorganic acids, bases, or amino acids, and including various forms of citrate, phosphate, tartrate, succinate, adipate, maleate, lactate, acetate, bicarbonate, or carbonate ions. Particularly advantageous buffers for use in parenterally administered forms of the presently disclosed compositions in the present disclosure include sodium or potassium buffers, including sodium phosphate, potassium phosphate, sodium succinate and sodium citrate.

Sodium chloride can be used to modify the tonicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine, arginine, can be included as 0-0.05% polysorbate-80 (optimally 0.005-0.01%).

In one embodiment, sodium phosphate is employed in a concentration approximating 20 mM to achieve a pH of approximately 7.0. A particularly effective sodium phosphate buffering system comprises sodium phosphate monobasic monohydrate and sodium phosphate dibasic heptahydrate. When this combination of monobasic and dibasic sodium phosphate is used, advantageous concentrations of each are about 0.5 to about 1.5 mg/ml monobasic and about 2.0 to about 4.0 mg/ml dibasic, with preferred concentrations of about 0.9 mg/ml monobasic and about 3.4 mg/ml dibasic phosphate. The pH of the formulation changes according to the amount of buffer used.

Depending upon the dosage form and intended route of administration it may alternatively be advantageous to use buffers in different concentrations or to use other additives to adjust the pH of the composition to encompass other ranges. Useful pH ranges for compositions of the present disclosure include a pH of about 2.0 to a pH of about 12.0.

In some embodiments, the pharmaceutical composition further comprises an adjuvant. Examples of adjuvants include, but are not limited to, water-in-oil or oil-in-water emulsions (e.g. Freund's adjuvant (complete and incomplete), MONTANIDE™ ISA 51, MONTANIDE™ ISA 720 VG MONTANIDE™ ISA 50V, MONTANIDE™ ISA 206, MONTANIDE™ IMS 1312, MF59® and AS03), aluminum salts (e.g. aluminum hydroxide, aluminum phosphate and potassium aluminum sulfate (also referred to as Alum)), liposomes, virosomes, 3-O-desacyl-4′-monophosphoryl lipid A (MPL) and adjuvants containing MPL (e.g. AS01, AS02 and AS04); saponin-based adjuvants, including saponin-based adjuvants (e.g., iscoms, iscom matrix, ISCOMATRIX™ adjuvant, MATRIX-M™ adjuvant, MATRIX-C™ adjuvant, Matrix Q™ adjuvant, ABISCO™-100 adjuvant, ABISCO™-300 adjuvant, ISCOPREP™ adjuvants and derivatives, including QS-21 and QS-21 derivatives); saponin derivatives from, e.g., Quillaja saponaria, Panax ginseng, Panax notoginseng, Panax quinquefolium, Platycodon grandiflorum, Polygala senega, Polygala tenuifolia, Quillaja brasiliensis, Astragalus membranaceus and Achyranthes bidentate; polyamino acids, co-polymers of amino acids, saponin, paraffin oil, muramyl dipeptide, Regressin (Vetrepharm, Athens GA), Avridine, liposomes, oil, Corynebacterium parvum, Bacillus Calmette Guerin, iron oxide, sodium alginate, unmethylated CpG motifs, glucan, 3 De-O-acylated monophosphoryl lipid A (MPL), MF59 (Novartis), AS03 (GlaxoSmithKline), AS04 (GlaxoSmithKline), and dextran sulfate. Additionally, CTL responses can be primed by conjugating one or more of the protein immunogens to lipids, such as tripalmitoyl-S-glycerylcysteinyl-seryl-serine (P3CSS). In certain embodiments, the vaccine composition includes QS-21 at 50 μg/dose/subject.

The pharmaceutical composition of the present application can be stored as a lyophilized powder under aseptic conditions and combined with a sterile aqueous solution prior to administration. The aqueous solution can contain pharmaceutically acceptable auxiliary substances as required to approximate physical conditions, such as pH adjusting and buffering agents, tonicity adjusting agents and the like, as discussed above. Alternatively, the pharmaceutical composition of the present application can be stored as a suspension, preferable an aqueous suspension, prior to administration.

The pharmaceutical composition of the present disclosure is formulated to be compatible with its intended route of administration. Examples of routes of administration include percutaneous and subcutaneous administration. Solutions or suspensions used for subcutaneous application can include the following components: a sterile diluent such as water for injection, saline solutions, fixed oils, polyethylene glycols, glycerin; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The compositions may be enclosed in ampoules, disposable syringes or multiple dose vials made of glass or plastic.

It is especially advantageous to formulate the pharmaceutical composition of the present application in dosage unit form for ease of administration and uniformity of dosage.

The present disclosure is further illustrated by the following examples that should not be construed as limiting. The contents of all references, patents, and published patent applications cited throughout this application, as well as the Figures and Tables, are incorporated herein by reference.

### EXAMPLES

### Example 1: Materials and Methods

### Cells

BSC-40 cells were obtained from ATCC and maintained in Dulbecco's Modified Eagle's Medium (DMEM) supplemented with 10% fetal bovine serum (FBS). RK13 cells were obtained from ATCC and maintained in Earle's Minimum Essential Medium (EMEM) supplemented with 10% FBS.

### Viruses and Infections.

vF13L-HA and vΔF13L were generously provided by Bernard Moss (National Institutes of Health, Bethesda), and their generation has been described previously (R. Blasco, B. Moss, Extracellular vaccinia virus formation and cell-to-cell virus transmission are prevented by deletion of the gene encoding the 37,000-Dalton outer envelope protein. Journal of virology 65, 5910-5920 (1991); M. Husain, B. Moss, Vaccinia virus F13L protein with a conserved phospholipase catalytic motif induces colocalization of the B5R envelope glycoprotein in post-Golgi vesicles. Journal of virology 75, 7528-7542 (2001); M. Husain, A. Weisberg, B. Moss, Topology of epitope-tagged F13L protein, a major membrane component of extracellular vaccinia virions. Virology 308, 233-242 (2003); T. R. Fuerst, E. G. Niles, F. W. Studier, B. Moss, Eukaryotic transient-expression system based on recombinant vaccinia virus that synthesizes bacteriophage T7 RNA polymerase. Proceedings of the National Academy of Sciences of the United States of America 83, 8122-8126 (1986)). vMC021L-HA has been described previously (S. R. Monticelli, P. Bryk, B. M. Ward, The Molluscum Contagiosum Gene MC021L Partially Compensates for the Loss of Its Vaccinia Virus Homolog F13L. Journal of virology 10.1128/jvi.01496-20, JVI.01496-01420 (2020)). vF13L-HA/A4L-mCherry and vΔF13L/A4-mCherry have been described previously (P. Bryk, M. G. Brewer, B. M. Ward, Vaccinia virus phospholipase protein F13 promotes the rapid entry of extracellular virions into cells. Journal of virology 10.1128/jvi.02154-17 (2018)). vMC021L-HA/A4L-mCherry was generated by infecting HeLa cells with vMC021L-HA followed by transfection with a plasmid expressing the red fluorescent protein, mCherry, fused to the N terminus of A4 with 500-bp flanking homology, and screened for by the production of red plaques. The MC021L/F13L locus was sequenced to verify its integrity. vF13L-HA/B5R-GFP/A4L-mCherry, vMC021L-HA/B5R-GFP/A4L-mCherry, and vΔF13L/B5R-GFP/A4L-mCherry were generated by infecting HeLa cells with vF13L-HA/A4L-mCherry, vMC021L-HA/A4L-mCherry, and vΔF13L/A4L-mCherry, respectively, followed by transfection with pB5R-GFP (B. M. Ward, B. Moss, Visualization of intracellular movement of vaccinia virus virions containing a green fluorescent protein-B5R membrane protein chimera. Journal of virology 75, 4802-4813 (2001); B. M. Ward, B. Moss, Golgi Network Targeting and Plasma Membrane Internalization Signals in Vaccinia Virus B5R Envelope Protein. Journal of virology 74, 3771-3780 (2000)) and screened for by the production of green and red plaques. VACV-GFP was previously described (C. C. Norbury, D. Malide, J. S. Gibbs, J. R. Bennink, J. W. Yewdell, Visualizing priming of virus-specific CD8+ T cells by infected dendritic cells in vivo. Nat Immunol 3, 265-271. (2002)).

### Mice.

C57BL/6 or Balb/c mice were purchased from Charles River Laboratories or Jackson Laboratories. Breeding pairs of STAT1+/−mice were purchased from Jackson Laboratories. bred in the specific-pathogen-free animal facility at the Penn State Hershey College of Medicine. For intradermal (i.d.) VACV infections, mice aged 7-10 weeks were anesthetized with ketamine/xylazine and injected with 104 PFU of VACV in <10 μL in each ear pinna. For footpad ECTV infections, mice were injected with 3000 PFU ECTV Moscow in the right footpad. During lethal challenge of STAT1−/− with VACV, or of Balb/c mice with ECTV, mice were monitored for death twice daily for 28 days following infection. To monitor pathogenesis in the ears, ear thickness was measured using a 0.0001 in. micrometer (Mitutoyo, Aurora, IL). Lesion progression and subsequent tissue loss were subsequently measured daily.

### Plaque Assays and Virus Quantification.

Plaque assays were conducted as previously described (S. R. Monticelli, P. Bryk, B. M. Ward, The Molluscum Contagiosum Gene MC021L Partially Compensates for the Loss of Its Vaccinia Virus Homolog F13L. Journal of virology 10.1128/jvi.01496-20, JVI.01496-01420 (2020)). Plaques were imaged after 3 days using a Leica DMIRB inverted fluorescence microscope with a cooled charge-coupled device (Cooke) controlled by Image-Pro Plus software (Media Cybernetics). Images were compiled and minimally processed using Photoshop (Adobe). Viral genomes were quantified by qPCR as described previously (J. L. Baker, B. M. Ward, Development and comparison of a quantitative TaqMan-MGB real-time PCR assay to three other methods of quantifying vaccinia virions. Journal of virological methods 196, 126-132 (2014)).

### Analysis of EEV.

EV purification by CsCl gradient was conducted as described previously (Monticelli 2020). Virus pellets were diluted in protein gel sample buffer, and analyzed by Western blotting as described previously (S. R. Monticelli, A. K. Earley, J. Tate, B. M. Ward, The Ectodomain of the Vaccinia Virus Glycoprotein A34 Is Required for Cell Binding by Extracellular Virions and Contains a Large Region Capable of Interaction with Glycoprotein B5. Journal of virology 93, e01343-01318 (2019)). The following antibodies were used: rabbit anti-HA antiserum (Sigma), rat anti-B5 MAb (M. Schmelz et al., Assembly of vaccinia virus: the second wrapping cisterna is derived from the trans Golgi network. Journal of virology 68, 130-147 (1994)), rabbit anti-A33 antiserum (NR-628; BEI Resources), mouse anti-actin MAb (Sigma), and rabbit anti-L1 antiserum (NR-631; BEI-Resources). HRP- and Alexa Fluor 647-conjugated donkey anti-rat and anti-mouse antibodies were purchased from Jackson ImmunoResearch Laboratories. HRP was detected using chemiluminescent reagents (Pierce) following the manufacturer's instructions. The fluorescent and chemiluminescent signal was captured using a Kodak Image Station 4000 mm Pro (Carestream Health Inc.).

### Flow Virometry.

RK13 cells were infected with the indicated viruses at a MOI of 5 at 37° C. The following day, cell culture supernatants were collected and clarified by low-speed centrifugation at 913×g for 10 min. Clarified supernatants were mixed with filtered 100% ethanol to a final concentration of 70% ethanol and incubated overnight at room temperature, and the next day, were clarified by low-speed centrifugation at 913×g for 10 min. Clarified supernatants were overlaid on a 36% sucrose cushion and centrifuged at 100,000×g for 40 min to pellet EV. EV were resuspended in Tris-EDTA (TE) buffer containing primary antibodies, as described in the figure legends, and incubated for 3 h at 4° C. EV were then pelleted at 16,000×g for 10 min, washed three times with TE buffer, and resuspended in TE buffer containing secondary antibodies, as described in the figure legends, and incubated at 4° C. 3 hrs later, EV were pelleted, washed, and resuspended in TE buffer as described above. Stained EV were analyzed with an LSRII-18 color BD Biosciences flow cytometer, using appropriate lasers and filters. Virions were separated from debris by gating for mCherry+events (A4-mCherry) and mCherry+events were gated for Cy2 (HA or B5-GFP), Alexa Fluor 647 (A33 or HA), Dylight 405 (B5), and Alexa Fluor 750 (A33) positive events, as described in the figure legends. The following antibodies were used: rabbit anti-HA antiserum (Sigma), rat anti-B5 MAb (12), and mouse anti-A33 MAb (NR-49231; BEI Resources). Alexa Fluor 647-conjugated donkey anti-mouse and anti-rabbit antibodies, Dylight 405-conjugated donkey anti-rat antibody, and Cy2-conjugated donkey anti-rabbit antibody were purchased from Jackson ImmunoResearch Laboratories. Alexa Fluor 750-conjugated goat anti-mouse antibody was purchased from Invitrogen. All data were analyzed using FACSDiva 8.0.1 (BD Biosciences) and FCS Express 7 (De Novo Software).

### Fluorescence Activated Virion Sorting.

RK13 cells were infected with the indicated viruses at a MOI of 5 at 37° C. The following day, cell culture supernatants were collected and clarified by low-speed centrifugation at 913×g for 10 min, overlaid on a 36% sucrose cushion, and centrifuged at 100,000×g for 40 min to pellet EV. EV were resuspended in TE buffer and sorted in a BSL-2 facility with a BD FACSAria II flow cytometer, using appropriate lasers and filters. Positive virions were first isolated by gating for mCherry+events (A4-mCherry) through a 610/20 bandpass filter and sorted based on high, medium, and low GFP fluorescence emission through a 525/50 bandpass filter. Sorted EV were pelleted at 16,000×g for 10 min and resuspended in TE buffer for qPCR, binding, and plaque assays.

### Cell Binding.

A virus binding assay was performed and quantified by qPCR as described previously (P. Bryk, M. G. Brewer, B. M. Ward, Vaccinia virus phospholipase protein F13 promotes the rapid entry of extracellular virions into cells. Journal of virology 10.1128/jvi.02154-17 (2018); Monticelli 2020, Monticelli 2019, S. R. Monticelli, A. K. Earley, R. Stone, C. C. Norbury, B. M. Ward, Vaccinia Virus Glycoproteins A33, A34, and B5 Form a Complex for Efficient Endoplasmic Reticulum to trans-Golgi Network Transport. Journal of virology 94, e02155-02119 (2020); J. L. Baker, B. M. Ward, Development and comparison of a quantitative TaqMan-MGB real-time PCR assay to three other methods of quantifying vaccinia virions. Journal of virological methods 196, 126-132 (2014)).

### Neutralizing Antibody.

A flow cytometric assay for measuring neutralizing antibody titiers has been previously described (P. L. Earl, J. L. Americo, B. Moss, Development and use of a vaccinia virus neutralization assay based on flow cytometric detection of green fluorescent protein. J Virol 77, 10684-10688 (2003)).

### Intracellular Cytokine Staining Assay.

Single cell suspensions generated from spleen, lymphocytes were isolated by centrifugation over Lymphocyte Separation Medium (Cambrex) and then stimulated for 4 h with 1 μM of each VACV peptide prior to the addition of 10 μg/mL brefeldin A (BFA; Sigma). VACV-derived peptides B8, A8, A3, K3, A47 and A42 have been previously described (A. R. Hersperger, N. A. Siciliano, B. C. DeHaven, A. E. Snook, L. C. Eisenlohr, Epithelial immunization induces polyfunctional CD8+ T cells and optimal mousepox protection. J Virol 88, 9472-9475 (2014)). Following peptide stimulation, cells were blocked in 2.4G2 supernatant containing 10% mouse normal mouse serum and then stained for CD8. Cells were fixed in 2% paraformaldehyde then permeabilized and stained for intracellular IFN-γ in 2.4G2 supernatant supplemented with 10% normal mouse serum and 0.5% saponin. Net frequencies and numbers of cytokine-positive TCD8+ were calculated by subtracting the unstimulated background response.

### Example 2: Expression of MC021-HA Results in EV with Increased Quantities of Glycoprotein

EV produced by vMC021L-HA incorporates more MC021-HA compared to its homolog, F13-HA. Glycoproteins, A33, A34, and B5, play multiple roles in EV target cell binding and outer membrane dissolution, and deletion or alteration of the incorporation of these glycoproteins results in the production of EV that are less infectious. Considering that there are differences in the levels of F13/MC021, it is likely that vMC021-HA may have altered the glycoprotein content of EV, leading to their decreased infectivity.

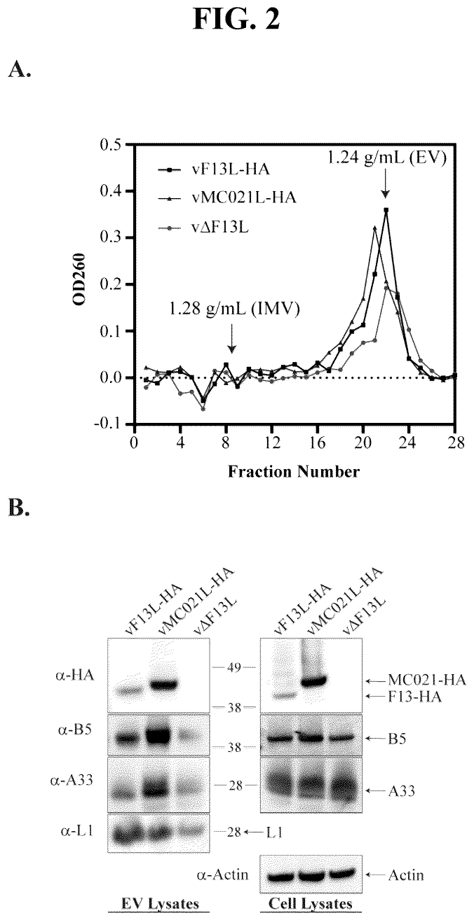

To determine if B5 and A33 were incorporated into the envelope of EV, EV released from cells infected with vF13L-HA/A4L-mCherry, vMC021L-HA/A4L-mCherry, and vΔF13L/A4L-mCherry (referred to as vF13L-HA, vMC021L-HA, and vΔF13L, respectively) were collected and purified by CsCl gradient ultracentrifugation (FIG. 2). A single peak of virions at 1.24 g/ml, representing EV, was present for each virus. In contrast, little to no IMV were detected by OD260 at 1.28 g/ml of CsCl where IMV are known to fractionate at. Therefore, fractions at 1.24 g/ml of CsCl and on either side, comprising EV, were collected and the EV contained within were isolated and analyzed by Western blot (FIG. 2, Panel B). To equilibrate the amount of EV loaded, samples were initially normalized for the amount of the IMV protein L1. MC021-HA appeared to be incorporated to a greater extent than F13-HA. Similarly, EV produced by cells infected with vMC021L-HA also incorporated more B5 and A33 compared to EV produced by vF13L-HA and vΔF13L. Western blot of infected cell lysates confirmed expression of F13-HA/MC021-HA, A33, and B5 (FIG. 2, Panel B).

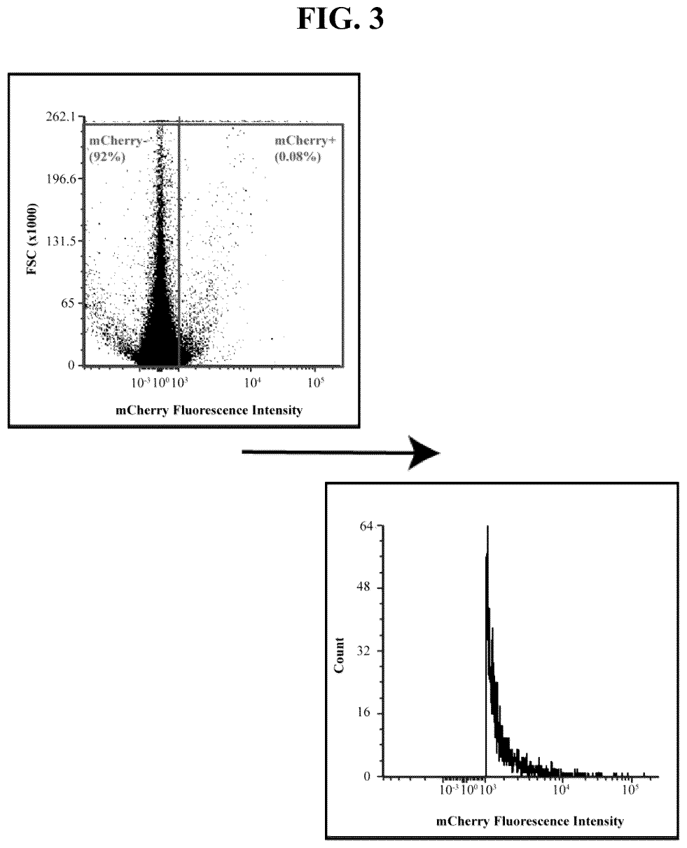

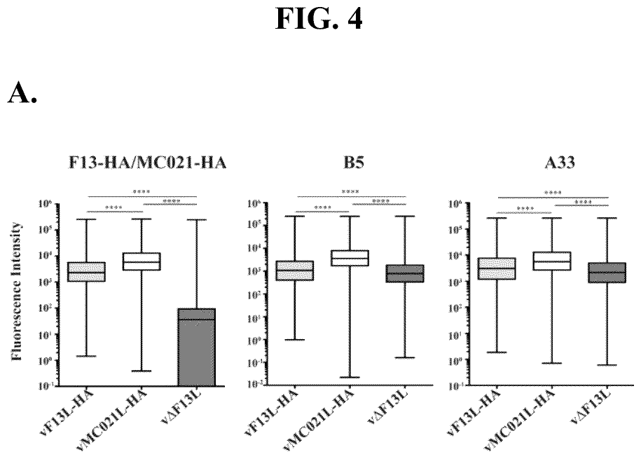

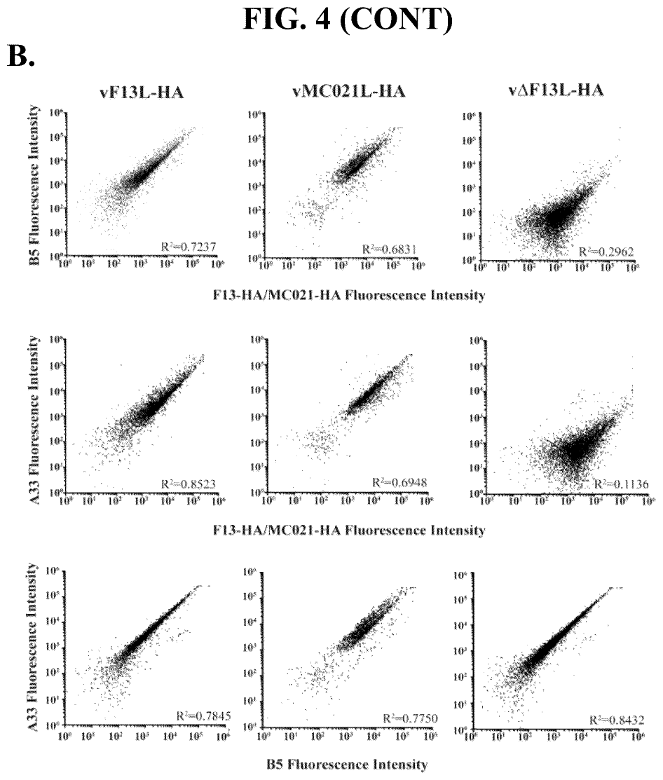

An increased amount of F13-HA/MC021-HA, A33, and B5 was incorporated in the outer membrane of EV produced by vMC021L-HA, even though this virus exhibits a small plaque phenotype (FIG. 2, Panel B). Therefore, a flow virometry assay was used to quantitatively examine glycoprotein incorporation for individual EV (FIG. 4). Using antibodies specific to A33, B5, and the HA epitope tag on either F13 or MC021, fixed VACV EV were fluorescently labeled and analyzed using flow cytometry. Of note, all of the recombinant viruses used express a core protein, A4, fused to mCherry. Thus, individual virions were initially differentiated from debris and extracellular vesicles by mCherry fluorescence (FIG. 3).

Next, mCherry+ virions were examined for levels of F13-HA/MC021-HA (HA), A33, and B5. Consistent with FIG. 2, EV produced by vMC021L-HA incorporated approximately twice as much B5 and thrice as much A33 compared to EV produced by vF13L-HA and vΔF13L EV, (FIG. 4, Panel A). Similarly, more MC021-HA was found in EV than F13L-HA while little-to-no signal was detected for EV produced by vΔF13L (FIG. 4, Panel A). Surprisingly, EV produced by vF13L-HA and vΔF13L EV contained equal amounts of B5 and A33 signal, suggesting that the presence/absence of F13 did not affect the amount of B5 and A33 incorporated.

To better understand the relationship between the detected proteins and their increased incorporation, EVs were plotted for their HA signals (x axis), i.e., F13 or MC021, against their A33 signals (y axis) (FIG. 4, Panel B, top row), HA against B5 (FIG. 4, Panel B, middle row), and A33 against B5 (FIG. 4, Panel B, bottom row). Correlation values (R2) were calculated to determine if there was a linear relationship for protein incorporation. In general, correlation values for B5:HA, B5:A33, and A33:HA was high for both vF13L-HA and vMC021L-HA indicating that for each increase in one of the proteins incorporated, there was an equal increase for the other two proteins. This linear relationship indicates that stoichiometric amounts of these proteins are incorporated during envelopment. In contrast, there was a low R2 value for the pairwise comparisons of HA signal to either B5 or A33 for vΔF13L (FIG. 4, Panel B; right column, top and middle rows). Interestingly, the best correlation was seen for B5:A33 in virions produced by vΔF13L. Considering the correlation values were fairly similar between vF13L-HA and vMC021L-HA, then MC021-HA increases protein incorporation overall but does not change the stoichiometric relationship of glycoprotein incorporation.

The increase in protein incorporation for vMC021L-HA EV is the result of virion aggregation that would result in the simultaneous analysis of multiple virions (coincidental analysis), rather than a single virion. The amount of the core protein should not vary; therefore, the mCherry signal was plotted against HA, B5, and A33 to ascertain whether the detected increase in protein incorporation was the result of virion aggregation. For each virus, the mCherry signal had little to no significant correlation with B5, A33, or HA (Table 1) indicating that the level of these proteins was independent of the amount of core protein present. Therefore, virion aggregation does not account for the large differences in HA, B5, or A33 incorporation between the viruses and the increase in protein incorporation for EV produced by vMC021L-HA can be attributed to the expression of MC021-HA.

TABLE 1
Correlation of glycoprotein signal with core mCherry

| | | Pearson correlation |
|---|---|---|
| Virus | Protein | coefficient (R²) |
| vF13L-HA | HA vs. mCherry | 0.0001382 |
| | B5 vs. mCherry | 0.0001253 |
| | A33 vs. mCherry | 0.0001836 |
| vMC021L-HA | HA vs. mCherry | 0.0000431 |
| | B5 vs. mCherry | 0.0004555 |
| | A33 vs. mCherry | 0.0004181 |
| vΔF13L | HA vs. mCherry | 0.0000027 |
| | B5 vs. mCherry | 0.0000109 |
| | A33 vs. mCherry | 0.0000083 |

This application discloses that F13 controls the amount of glycoprotein incorporated into the envelope of EV during intracellular envelopment; F13 limits glycoprotein incorporation while the MOCV homolog facilitates greater incorporation. Concurrently, there is an increase in the amount of MC021 found in the envelope (FIG. 2, Panel B and FIG. 4, Panel A). The strong correlation between the amount of A33, B5, and F13/MC021 found in each virion indicates a stoichiometric relationship between their incorporation, indicating that at least these three proteins are incorporated as a complex. Even though F13L-HA and MC021L-HA were both expressed from an identical F13L promoter, a greater amount of MC021-HA was detected in lysates from infected cells compared to F13-HA (FIG. 2, Panel B). The increased level of MC021 could also account for the increased envelope incorporation by facilitating interactions between EV proteins and proteins on the surface of IMV. It is highly likely that at least one of the four critical EV proteins (A33, A34, B5, and F13) if not all, serves as a matrix-like protein to facilitate envelopment of IMV to form EV. Thus, an increase in formation of a matrix-like complex should result in increased interactions between EV and IMV proteins, accounting for their increased incorporation into released virions.

### Example 3: B5-GFP can Functionally Replace B5 for EV Production

A decrease in A33, A34, and/or B5 incorporation correlates with a decrease in the infectivity of EV and a small plaque phenotype. However, this is the first time an increase in glycoprotein incorporation has correlated to a defect in EV infectivity. These results suggest that there is an optimal glycoprotein concentration required for optimal EV infectivity.

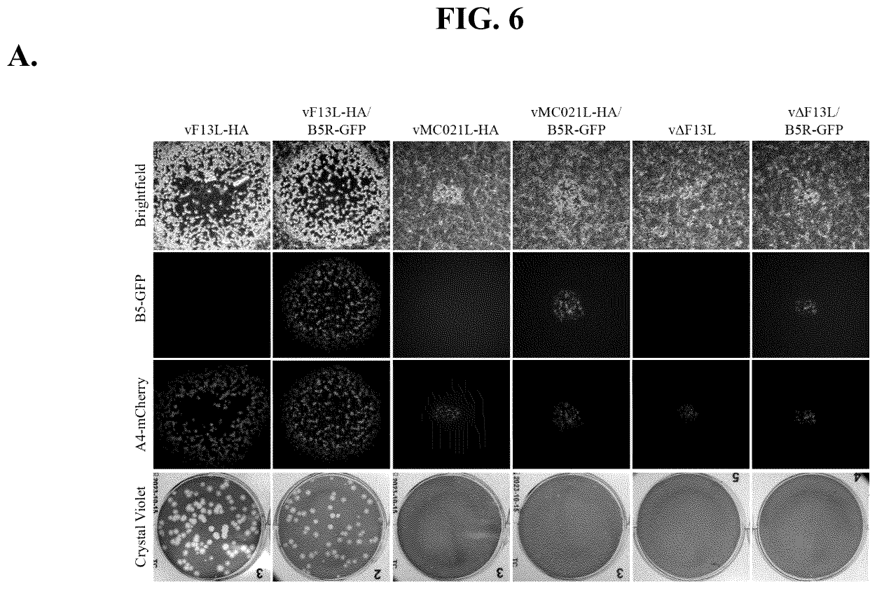

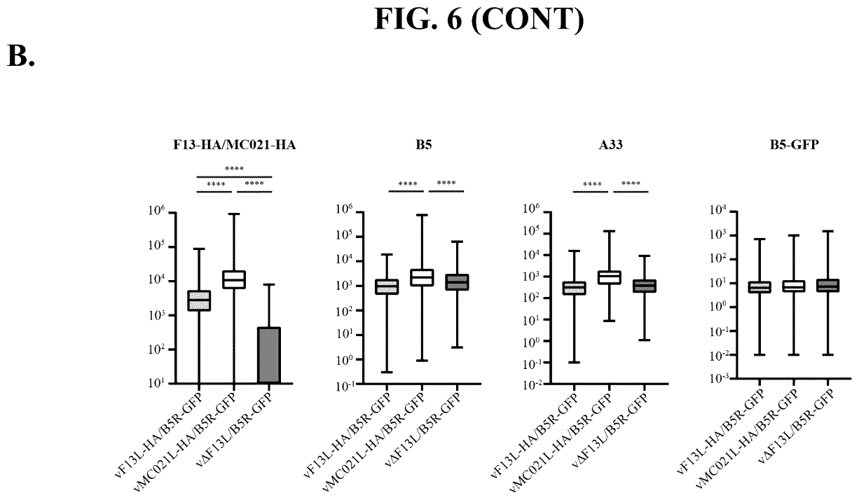

To test this hypothesis, the B5R gene in the three recombinant viruses was replaced with B5R-GFP (now termed vF13L-HA/B5R-GFP, vMC021L-HA/B5R-GFP, and vΔF13L/B5R-GFP) to monitor the levels of the glycoprotein B5 in released EV without the use of antibodies. The addition of GFP to B5 can functionally replace B5 during infection and B5-GFP has proven to be a useful tool for studying IEV egress in living cells. To verify that B5-GFP could functionally replace B5 in the context of vMC021L-HA infection, the plaque phenotypes of the A4L-mCherry parental viruses were compared to the new recombinants that express B5-GFP in place of B5 (FIG. 6, Panel A). Comparison of the plaque phenotypes of these viruses revealed no difference between vF13L-HA and vF13L-HA/B5R-GFP, vMC021L-HA and vMC021L-HA/B5R-GFP, and vΔF13L and vΔF13L/B5R-GFP, suggesting that B5-GFP can functionally replace B5 during infection.

Unexpectedly, flow virometric analysis revealed a large range of protein incorporation per virion for all three of the recombinant viruses analyzed, suggesting a stochastic process for envelope composition (FIGS. 4 and 6). Furthermore, it is the increase in glycoprotein content that leads to a decrease in cell binding and an overall reduction in the infectivity of vMC021L-HA. It is generally accepted that in multivalent systems, such as virion glycoproteins interacting with cellular receptors, an increase in the number of ligands (glycoproteins) on the surface of the particle should lead to an increase in binding affinity. It is unexpected to find that an overall increase in glycoprotein density on the virion surface was equally detrimental to cell binding.

### Example 4: The Addition of GFP to B5 does not Impact the Incorporation Profile of HA, A33, and B5

The expression of MC021-HA can impact the incorporation of B5-GFP relative to B5. To determine whether the incorporation profile of the A4L-mCherry/B5R-GFP recombinants looked similar to the incorporation profile of the A4L-mCherry parental viruses (FIG. 4), EV produced by the new recombinant viruses were analyzed by flow virometry. Analysis of the data shows that an incorporation profile similar to the A4-mCherry parental viruses was observed for the B5-GFP viruses (FIG. 6, Panel B) with vMC021L-HAB5R-GFP EV incorporating approximately 2-3-fold more HA, A33, and B5 compared to vF13L-HAB5R-GFP. Furthermore, correlation plots were generated where B5 signal (x axis) was plotted against A33 signal (y axis), HA against B5, and A33 against HA (Table 2). Correlation values (R2) were calculated and were found to be similar to results with the parental viruses (FIG. 4, Panel B). Altogether, these results indicated that the B5-GFP viruses resembled the parental viruses in both functionality and pattern of glycoprotein incorporation. GFP fluorescence was not detected in these fixed samples, likely due to the denaturation of GFP by the ethanol fixation protocol used.

TABLE 2
Correlations of detected signal for B5-GFP recombinant viruses.

| | | Pearson | |
|---|---|---|---|
| | | Correlation | |
| Virus | Protein | Coefficient (R²) | Significance^{a} |
| vF13L-HA/B5R-GFP | A33 vs. B5 | 0.8041 | **** |
| | B5 vs. HA | 0.8750 | **** |
| | A33 vs. HA | 0.7856 | **** |
| vMC021L-HA/B5R-GFP | A33 vs. B5 | 0.7005 | **** |
| | B5 vs. HA | 0.6150 | **** |
| | A33 vs. HA | 0.6993 | **** |
| vΔF13L/B5R-GFP | A33 vs. B5 | 0.9434 | **** |
| | B5 vs. HA | 0.2186 | N/A |
| | A33 vs. HA | 0.2560 | N/A |

### Example 5: EV Containing an Intermediate Level of B5-GP have a Higher Specific Infectivity Compared to EV with Either Higher or Lower B5-GFP Content

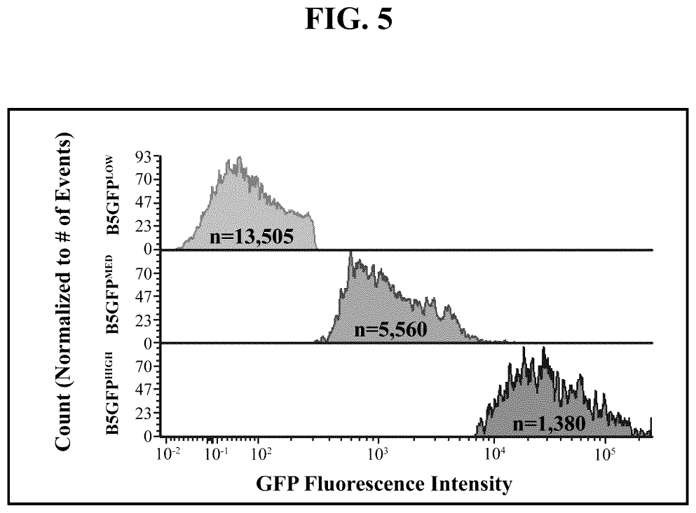

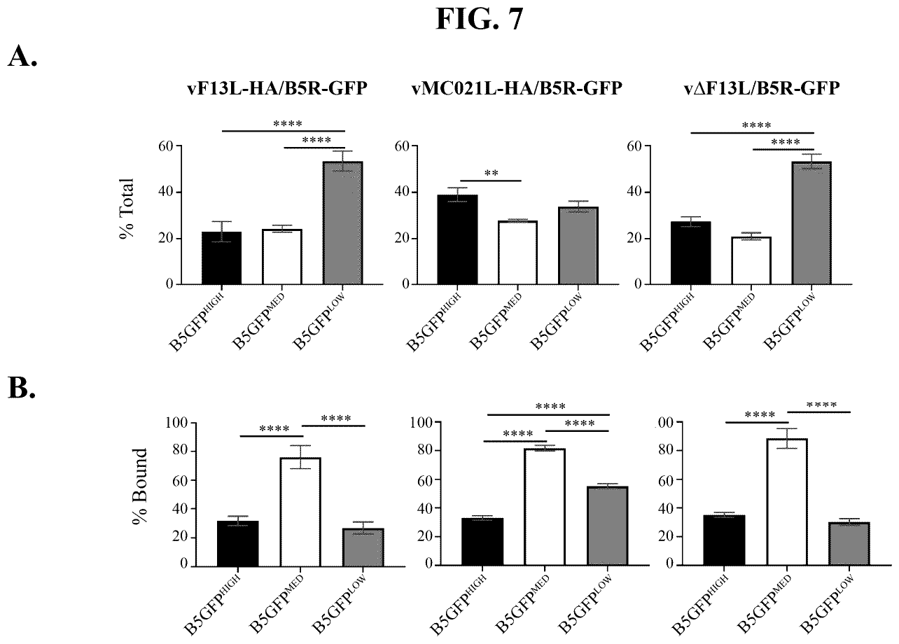

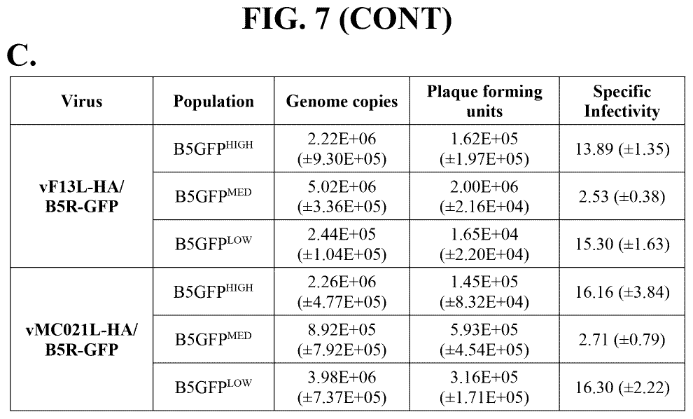

After verifying that the B5-GFP viruses recapitulate the plaque phenotype and incorporation profile of the parental A4-mCherry viruses (FIG. 6), a fluorescence activated virion sorting (FAVS) experiment was conducted using unfixed EV. As above, EV were identified by gating on mCherry+ events and then sorted based on high, medium, or low GFP fluorescence intensity (termed B5GFP^{HIGH}, B5GFP^{MED}, and B5GFP^{LOW}, respectively; FIG. 5). Although sorting was conducted according to their B5-GFP content, based on the data in FIG. 4, Panel B, the levels of A33 and F13/MC021 should correlate with the level of B5-GFP. Following EV sorting, the infectious properties of these three groups were investigated (FIG. 7).

Given that vMC021L-HA/B5R-GFP EV incorporate more B5 compared to both vF13L-HA/B5R-GFP and vΔF13L/B5R-GFP EV, it was expected that a larger percentage of the total amount of vMC021L-HA/B5R-GFP EV would fall within B5GFP^{HIGH}. To test this, the percentage of the total number of viral genome copies that comprised B5GFP^{HIGH}, B5GFP^{MED} and B5GFP^{LOW} were quantified by qPCR (FIG. 6, Panel A). For both vF13L-HA/B5R-GFP and vΔF13L/B5R-GFP, approximately 50% of the EV were either B5GFP^{HIGH} or B5GFP^{MED} and the other 50% was B5GFP^{LOW}. However, as expected, for vMC021L-HA/B5R-GFP, the proportion of EV that were B5GFP^{LOW} shifted to B5GFP^{HIGH} and B5GFP^{MED}, whereas approximately 70% of the EV were either B5GFP^{HIGH} or B5GFP^{MED} and only 30% was B5GFP^{LOW}.

Next, EV from B5GFP^{HIGH}, B5GFP^{MED}, and B5GFP^{LOW} were tested for cell binding using a sensitive qPCR-based binding assay (FIG. 7, Panel B). For vF13L-HA/B5R-GFP, B5GFP^{MED} EV exhibited the highest binding efficiency of approximately 76%, whereas B5GFP^{HIGH} and B5GFP^{LOW} EV displayed a significant reduction in binding efficiency compared to B5RGFP^{MED} of approximately 45-50%. A similar pattern was observed for both vΔF13L/B5R-GFP EV and vMC021L-HA/B5R-GFP EV, although in the latter case B5GFP^{LOW} EV exhibited a significant reduction of approximately 25% in binding efficiency compared to B5RGFP^{MED}. These results suggest that irrespective of the recombinant virus, EV containing an intermediate amount of B5 were better at binding cells than EV that have larger or smaller amounts of the glycoprotein.

Specific infectivity was calculated for B5GFP^{HIGH}, B5GFP^{MED}, and B5GFP^{LOW} EV produced by vF13L-HA/B5R-GFP and vMC021L-HA/B5R-GFP by comparing the total number of genome copies to plaque forming units (PFUs), (FIG. 7, Panel C). Whereas 1 out of every 2.53 genome copies resulted in a plaque for B5GFP^{MED} EV for vF13L-HA/B5R-GFP, this number was elevated almost 5-fold to 1 out of every 13.89 and 15.30 for B5GFPLOW and B5GFP^{HIGH} EV, respectively. Similarly, for vMC021L-HA/B5R-GFP, compared to B5GFP^{MED} EV, where 1 out of every 2.71 genome copies resulted in a plaque, B5GFPLOW and B5GFP^{HIGH}EV exhibited an approximate 6-fold increase in specific infectivity, whereas 1 out of every 16.16 and 16.20 genome copies resulted in a plaque, respectively. Importantly, the specific infectivity data correlated with the binding data. Altogether, this data shows that there is a direct relationship between EV glycoprotein incorporation and infectivity, and furthermore, that an optimal or “just right” amount of glycoprotein must be incorporated to produce highly infectious EV, irrespective of the recombinant virus used.

### Example 6: vMC021L-HA is Attenuated, but Retains Immunogenicity, In Vivo

vMC021L-HA produced as much EV as vF13L-HA, but exhibits a small plaque phenotype (FIG. 6, Panel A). Moreover, vMC021L-HA EV have increased amounts of glycoproteins A33 and B5, both of which have been shown to be the targets of neutralizing antibodies. Given these results, it was hypothesized that vMC021L-HA would be attenuated in vivo, but that the release of antigen may nonetheless allow generation of protective immune responses.

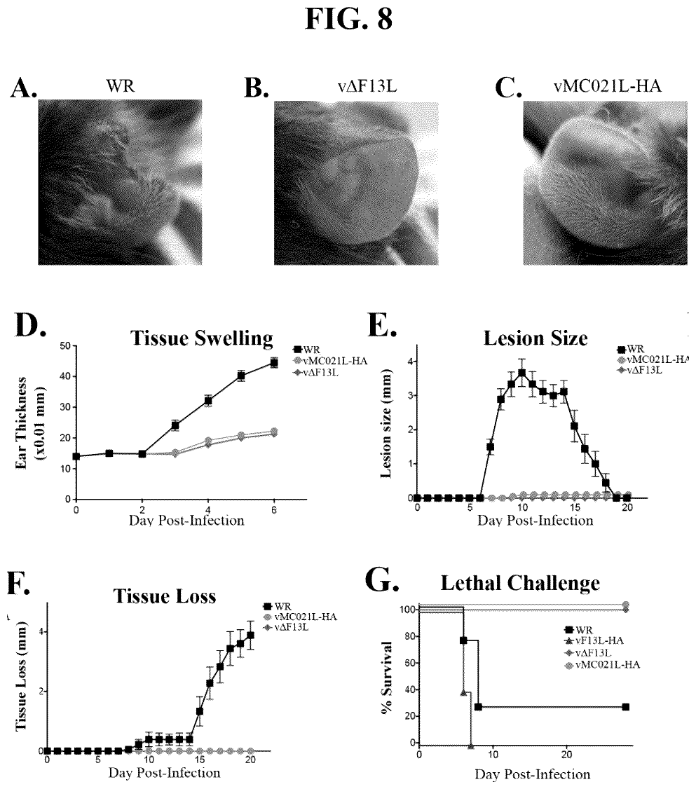

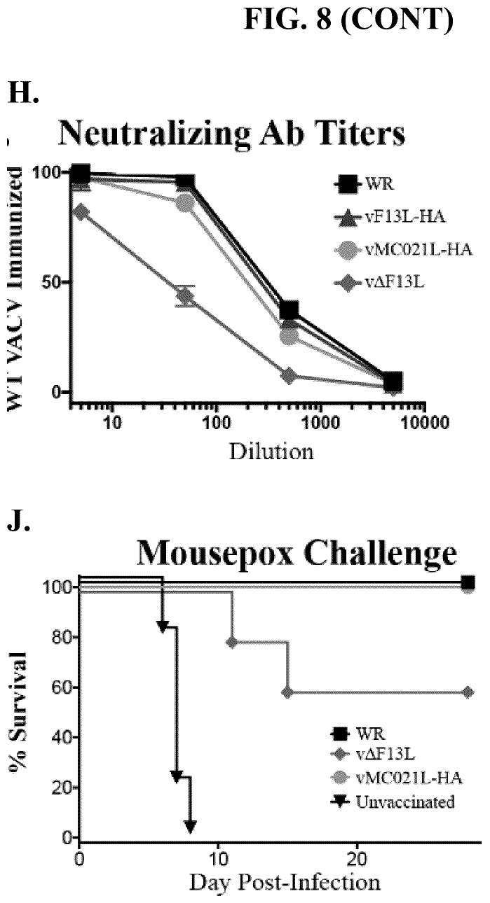

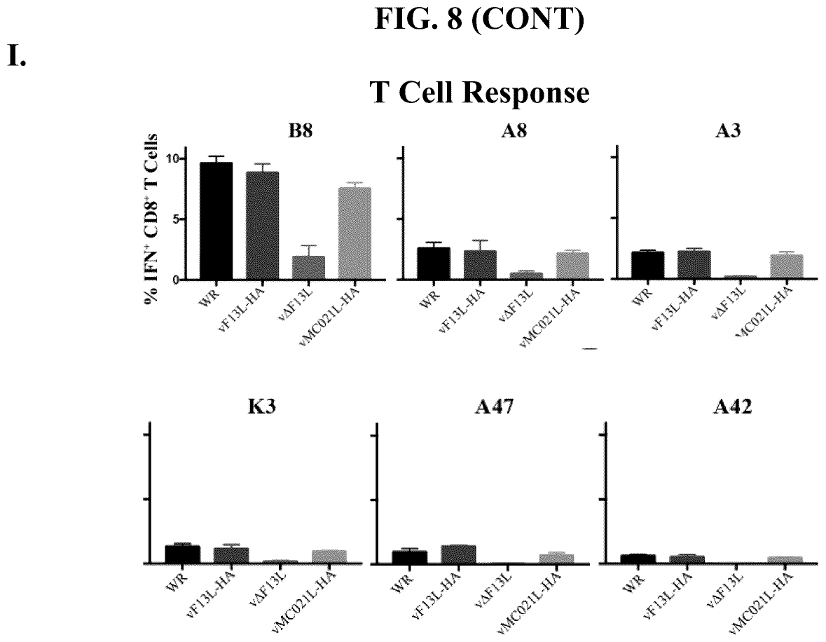

To test this hypothesis, wild-type mice were infected intradermally, a route that mimics both the natural route of infection and the route of immunization with vaccinia in the smallpox virus eradication program. 14 days after intradermal infection, tissue pathology could be readily identified in mice infected with VACV WR (Western Reserve) (FIG. 8, Panel A), but no pathology was readily observable after infection with either vΔF13L (FIG. 8, Panel B) or vMC021L-HA (FIG. 8, Panel C).

To quantify this, measurements were made of the characteristic swelling (FIG. 8, Panel D), lesion development (FIG. 8, Panel E) and tissue loss (FIG. 8, Panel F) normally associated with dermal VACV infection. Although infection with either vΔF13L or vMC021L-HA did trigger some local tissue swelling, this was much reduced compared to infection with VACV WR, and no observation was made of development of a lesion, or subsequent tissue loss after infection with either vΔF13L or vMC021L-HA. STAT1−/− mice are particularly sensitive to orthopoxvirus infection, and it was found that this sensitivity extends to cutaneous infection with limited doses of virus (FIG. 8, Panel G). Following infection with vF13L-HA, all STAT1−/− mice died by 8 days post-infection. However, all mice infected with either vMC021L-HA or vΔF13L-HA survived to the end of the experiment at 28 days post-infection. Therefore, vMC021L-HA and vΔF13L-HA are attenuated in vivo and that the increase in EV glycoprotein exhibited by vMC021L-HA contributes to this attenuation.

To assess whether the decrease in EV infectivity caused a commensurate reduction in immunogenicity in vivo, wild-type mice were infected intradermally with VACV WR, vF13L-HA, vΔF13L or vMC021L-HA and harvested serum 38d after infection. The ability of dilutions of serum from each cohort of infected animals to block VACV-GFP in vitro was examined. Immunization with either VACV WR or vF13L-HA produced serum antibodies that blocked 50% of infection at a dilution of ˜1:250, whereas immunization with vΔF13L only produced serum antibodies that blocked 50% of infection at a dilution of ˜1:30 (FIG. 8, Panel H). However, consistent with the increased levels of EV glycoproteins in vMC021L-HA, it was found that immunization with this virus produced serum antibodies that blocked 50% of infection at levels only just below those immunized with VACV WR, at a dilution of ˜1:200 (FIG. 8, Panel H). A potent TCD8+ response, that targets multiple epitopes, including the B8, A8, A3, K3, A47 and A42 epitopes in mice on an H2b background, is also induced by VACV WR 8 days after infection (FIG. 8, Panel I). A similar T cell response is observed after immunization with vF13L-HA, but immunization with vΔF13L induced a much-reduced response to all the determinants examined and was undetectable above background to the A47 and A42 epitopes (FIG. 8, Panel I).

In contrast, although it was assumed that the immunogenicity of the vMC021L-HA would only be enhanced to the EV glycoproteins, it was observed a T cell response to all determinants that was markedly above that induced following immunization with the vΔF13L, and often approached that observed after immunization with VACV WR or vF13L-HA. Therefore, the immunogenicity of vMC021L-HA appears intact, despite both a marked diminution in spread in vitro and induction of pathology in vivo. To assess the functionality of the correlates of protective immunity, susceptible Balb/c mice were immunized with VACV WR, vΔF13L or vMC021L-HA and challenged with the virulent mouse poxvirus ectromelia (ECTV) 35 days later. Mice that had not been immunized all died within 8 days of ECTV infection, while 40% of those immunized with vΔF13 died between day 10 and 20 post-infection, a time point that likely indicates a failure of the adaptive immune system to contain the virulent infection. However, mice immunized with either VACV WR or vMC021L-HA all survived the challenge, indicating the induction of functional protective immunity by vMC021L-HA.

This application demonstrates that F13 limits the relative amounts of EV glycoproteins incorporated in the outer EV membrane, and that there is an optimal concentration of glycoproteins required for infectivity. The unexpected side effect of changing the concentration of EV glycoproteins in the outer membrane is that immunogenic virions are produced from infected cells in normal quantities, and that these virions may display enhanced levels of EV proteins that are targets for a neutralizing antibody response. The result is a recombinant VACV that does not cause any discernable pathology upon immunization, but which produces neutralizing antibodies at levels approaching those induced upon infection with wild-type VACV. Unexpectedly, this vector also induced strong TCD8+ responses, a hallmark of VACV that spreads effectively. However, immunization with inactivated VACV virions can induce an effective TCD8+ response, so it is likely that the production of EV by cells infected at the site of immunization produces sufficient virus particles to reproduce, or even exceed this immunogenicity.

Therefore, careful manipulation of EV glycoprotein content can produce effective immunogenic viral vectors that do not display the side effects of traditional VACV vectors, but which display a marked enhancement in immunogenicity relative to non-replicating vectors.

While various embodiments have been described above, it should be understood that such disclosures have been presented by way of example only and are not limiting. Thus, the breadth and scope of the subject compositions and methods should not be limited by any of the above-described exemplary embodiments, but should be defined only in accordance with the following claims and their equivalents.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

1. A method for inducing an immune response to an antigen in a subject, comprising:
administering to the subject an effective amount of a recombinant vaccinia virus in which the extracellular virion protein F13 has been replaced with MC021, a molluscum contagiosum virus homolog of F13, wherein the recombinant vaccinia virus comprises a nucleic acid encoding an immunogenic epitope of the antigen.

2. The method of claim 1, wherein the antigen is a viral antigen.

3. The method of claim 2, wherein the viral antigen is an antigen from SARS-Cov-2.

4. The method of claim 1, wherein the antigen is a bacterial antigen.

5. The method of claim 1, wherein the antigen is a tumor antigen.

6. The method of claim 1, wherein the recombinant vaccinia virus is administered percutaneously, subcutaneously or intramuscularly.

7. A method for inducing a protective immune response to a target in a subject, comprising:
administering to the subject an effective amount of a recombinant vaccinia virus in which the extracellular virion protein F13 has been replaced with MC021, a molluscum contagiosum virus homolog of F13,
wherein the recombinant vaccinia virus comprises a nucleic acid encoding an immunogen from the target.

8. The method of claim 7, wherein the target is a microorganism.

9. The method of claim 8, wherein the microorganism is a virus.

10. The method of claim 9, wherein the virus is SARS-Cov-2.

11. The method of claim 8, wherein the microorganism is a bacterium.

12. The method of claim 7, wherein the target is a tumor cell.

13. The method of claim 7, wherein the recombinant vaccinia virus is administered percutaneously, subcutaneously or intramuscularly.

14. The method of claim 7, wherein the recombinant vaccinia virus is administered with an adjuvant.

15. A pharmaceutical composition comprising:
(a) a recombinant vaccinia virus in which the extracellular virion protein F 13 has been replaced with MC021, a molluscum contagiosum virus homolog of F13; and
(b) pharmaceutically acceptable carrier,
wherein the pharmaceutical composition is formulated for or percutaneous inoculation, subcutaneous injection or intramuscular injection.

16. The pharmaceutical composition of claim 15,
wherein the recombinant vaccinia virus comprises a nucleic acid encoding an immunogenic epitope of an antigen and is capable of expressing the epitope of the antigen upon infection of a target cell.

17. The pharmaceutical composition of claim 16, wherein the antigen is a viral antigen.

18. The pharmaceutical composition of claim 17, wherein the viral antigen is an antigen from SARS-CoV-2.

19. The pharmaceutical composition of claim 15,
wherein the recombinant vaccinia virus is an enveloped virus and wherein the virus envelop comprises, in addition to MC021, an additional foreign glycoprotein or a portion thereof, wherein the additional foreign glycoprotein is a viral protein from a different virus.

20. The pharmaceutical composition of claim 19, wherein the different virus is SARS-CoV-2.


SEQUENCE LISTING (2 sequences)

SEQ ID NO: 1 - protein, 372 aa, Vaccinia virus
  misc_feature: Vaccinia virus extracellular virion protein F13
MWPFASVPAGAKCRLVETLPENMDFRSDHLTTFECFNEIITLAKKYIYIASFCCNPLSTTRGALIFDKLKEASEKGIKIIVLLDERGKRNLGELQSHCPDINFITVNIDKKNNVGLLLGCFWVSDDERCYVGNASFTGGSIHTIKTLGVYSDYPPLATDLRRRFDTFKAFNSAKNSWLNLCSAACCLPVSTAYHIKNPIGGVFFTDSPEHLLGYSRDLDTDVVIDKLRSAKTSIDIEHLAIVPTTRVDGNSYYWPDIYNSIIEAAINRGVKIRLLVGNWDKNDVYSMATARSLDALCVQNDLSVKVFTIQNNTKLLIVDDEYVHITSANFDGTHYQNHGFVSFNSIDKQLVSEAKKIFERDWVSSHSKSLKI

SEQ ID NO: 2 - protein, 388 aa, Molluscum contagiosum virus
  misc_feature: MC021, a molluscum contagiosum virus homolog of F13
MGNLTSARPAGCKIVETLPATLPLALPTGSMLTYDCFDTLISQTQRELCIASYCCNLRSTPEGGHVLLRLLELARADVRVTIIVDEQSRDADATQLAGVPNLRYLKLDVGELPGGKPGSLLSSFWVSDKRRFYLGSASLTGGSISTIKSLGVYSECEPLARDLRRRFRDYERLCARRCVRCLSLSTRFHLRRHCENAFFSDAPESLIGSTRTFDADAVLAHVQAARSTIDMELLSLVPLVRDEDSVQYWPRMHDALVRAALERNVRVRLLVGLWHRSDVFSLAAVKGLHELGVGHADISVRVFAIPGAKGDAVNNTKLLVVDDEYVHVTSADMDGTHYARHAFVSFNCAERAFARALGALFERDWQSSFSSPLPRAPPPEPATLLPVN